(19) United States
(12) Patent Application Publication
(10) Pub. No.: US 2019/0328887 A1
(43) Pub. Date: Oct. 31, 2019

(54) METABOLICALLY STABLE PEPTIDE ANALOGS

(71) Applicants: UNIVERSITÉ DE STRASBOURG, STRASBOURG (FR); INSERM - INSTITUT NATIONAL DE LA SANTÉ ET DE LA RECHERCHE MÉDICALE, PARIS CEDEX 13 (FR); CENTRE NATIONAL DE LA RECHERCHE SCIENTIFIQUE, PARIS (FR); COLLÈGE DE FRANCE, PARIS (FR)

(72) Inventors: Dominique BONNET, GEISPOLSHEIM (FR); Catherine LLORENS CORTES, BURES-SUR-YVETTE (FR); Xavier ITURRIOZ, LA NORVILLE (FR)

(21) Appl. No.: 16/310,559

(22) PCT Filed: Jun. 16, 2017
(86) PCT No.: PCT/EP2017/064801

§ 371 Date: Dec. 17, 2018

(30) Foreign Application Priority Data
Jun. 16, 2016 (EP) 16305733.4

(51) Int. Cl. A61K 47/54; C07K 14/575; C07K 7/16; A61K 47/60

(52) U.S. Cl. CPC A61K 47/54; C07K 14/575; A61K 38/00; A61K 47/60; C07K 7/16

(57) ABSTRACT

The present invention relates to metabolically stable and non-immunogen analogs completely hydrosolubleat physiological pH, and their use for the prevention or treatment of diseases mediated by G-protein coupled receptor (GPCR), in particular (Central Nervous System) CNS and cardiovascular diseases or disorders, or their use in diagnostic methods.

### FIELD OF THE INVENTION

The invention relates to metabolically stable and non-immunogen analogs completely hydrosoluble, and their use for the prevention or treatment of diseases mediated by G-protein coupled receptor (GPCR), in particular (Central Nervous System) CNS and cardiovascular diseases or disorders, or their use in diagnostic methods.

### BACKGROUND OF THE INVENTION

To date more than 7000 naturally occurring peptides have been identified, and these often have a crucial role in human physiology including actions as hormones, neurotransmitters, growth factors, ion channel ligands, or anti-infectives. In general, peptides are selective and efficacious signaling molecules that bind to specific cell surface receptors, such as G-protein coupled receptors (GPCRs) or ion channels, where they trigger intracellular effects. Thereby, during the last decade, peptides have gained a wide range of applications in medicine. Indeed, more than 60 US Food and drug Administration (FDA)-approved peptide medicines are on the market and this is expected to grow significantly, with approximately 140 peptide drugs currently in clinical trials and more than 500 therapeutic peptides in preclinical development (Fosgerau, K.; Hoffmann, T. Peptide therapeutics: current status and future directions. Drug Discovery Today 2015, 20, 122-128) [1].

However, naturally occurring peptides are often not directly suitable for use as convenient therapeutics because they have intrinsic weaknesses, including poor chemical and physical stability, and a short circulating plasma half-life. (Vlieghe, P.; Lisowski, V.; Martinez, J.; Khrestchatisky, M. Synthetic therapeutic peptides: science and market. Drug Discovery Today 2010, 15, 40-56) [2].

The techniques developed for half-life extension are generally based on the modification of the native peptide backbone (non-natural aminoacids, lactam bridges, stapling, cyclization). Polyethylene glycol (PEG) incorporation into peptides has also been used to limit glomerular filtration and thereby increasing plasma half-life by limiting the elimination of peptides. However, because of increased safety and tolerability concerns relating to the use of PEG as a component of an injectable therapeutic, PEGylation has become a less preferred choice. Another approach is based on the binding of the peptide to the circulating protein albumin used as a vehicle by peptide acylation with hydrocarbon tail as seen in the GLP-1 agonist. Nevertheless the presence of the hydrocarbon tail makes the peptide less selective for its target with potential enhanced toxicity (Fosgerau, K.; Hoffmann, T. Peptide therapeutics: current status and future directions. Drug Discovery Today 2015, 20, 122-128) [1].

G-protein coupled receptors (GPCRs), also known as seven-transmembrane domain receptors, 7TM receptors, heptahelical receptors, serpentine receptors, and G protein-linked receptors (GPLR), constitute a large family of receptors, the G-protein-coupled receptor (GPCR) family (a superfamily/family within the transporter-opsin-G (TOG) protein-coupled receptors superfamily), that sense molecules outside the cell and activate inside signal transduction pathways and ultimately, cellular responses. Coupling with G proteins, they are called seven-transmembrane receptors because they pass through the cell membrane seven times. G protein-coupled receptors are found only in eukaryotes, including yeast, choanoflagellates, and animals. The ligands that bind and activate these receptors include light-sensitive compounds, odors, pheromones, hormones, and neurotransmitters, and vary in size from small molecules to peptides to large proteins. G protein-coupled receptors are involved in many diseases, and are also the target of approximately 40% of all modern medicinal drugs (Congreve, M.; Langmead, C. J.; Mason, J. S.; Marshall, F. H. Progress in Structure Based Drug Design for G Protein-Coupled Receptors. J. Med. Chem. 2011, 54, 4283-4311) [3].

As an example of GPCRs peptide, apelin serves important function in food intake, vasopressin and histamine release, gastric acid, bicarbonate secretion and insulin secretion, diuresis, cell proliferation, angiogenesis, glucose-fluid balance and regulation of gastrointestinal motility and cardiovascular system. (Narayanan S, Harris D L, Maitra R, Runyon S P. Regulation of the Apelinergic System and Its Potential in Cardiovascular Disease: Peptides and Small Molecules as Tools for Discovery. J Med Chem. 2015 Oct. 22; 58(20):7913-27) [4].

As another example of GPCRs peptide, angiotensin behaves as an antagonist of AT1R. It is well established that the clinical benefits of AT1R blockers extend far beyond their control of blood pressure, and include the reduction of mortality from myocardial infarction, heart failure, atherosclerosis, stroke, diabetes, peripheral vascular disease and chronic renal disease (Marc Y, Llorens-Cortes C. The role of the brain renin-angiotensin system in hypertension: implications for new treatment. Prog Neurobiol. 2011 October; 95(2):89-103) [5].

As another example of GPCRs peptide, there is growing evidence that the neuropeptide oxytocin (OT) modulates complex social behavior and social cognition. OT has been reported in human to improve prosocial behavior and trust, social memory, to decrease fear associated with social phobia (Modi, M. E.; Young, L. J. The oxytocin system in drug discovery for autism: Animal models and novel therapeutic strategies. Horm. Behav. 2012, 61, 340-350) [6].

However insofar as the half-life of apelin, angiotensin or oxytocin in the blood circulation is short, there is a need of designing, synthesizing and testing novel potent and more stable peptides derived therefrom that can activate the GPCR pathways.

### SUMMARY OF THE INVENTION

The invention relates to new fluorocarbon conjugates to increase therapeutic peptides half-life, in particular GPCR peptides half-life, thus leading to fluoropeptides metabolically more stable, non-immunogen and completely hydrosoluble at physiological pH. Such compounds constitute potential new therapeutic agents to prevent or treat diseases mediated by the GPCRs without increasing humoral and/or cellular immunogenicity to them.

In one aspect, the present invention relates to a metabolically stable and non-immunogenic peptide analog completely hydrosoluble at physiological pH having a peptide covalently linked to a fluorocarbon group, directly or through a linker selected from the group consisting of a PEG or a peptide having from 1 to 6 amino acids, either on the alpha-amino or the epsilon-amino group of at least one lysine of said peptide, when the linker is a lysine, the fluorocarbon group is directly linked to the epsilon-amino group of said linker;

but excluding an apelin analog having the following peptide of formula (I):

Lys-Phe-Xaa1-Arg-Xaa2-Arg-Pro-Arg-Xaa3-Ser-Xaa4-Lys-Xaa5-Pro-Xaa6-Pro-Xaa7 (I)

wherein said peptide of formula (I) (SEQ ID NO: 1) is linked to a fluorocarbon group, an acetyl group, or an acyl group —C(O)R where R is a C₇₋₃₀ alkyl, directly or through a linker selected from the group consisting of PEG, lysine and arginine, either on the alpha-amino or the epsilon-amino group of at least one lysine of the peptide formula (I), and when the linker is a lysine, the fluorocarbon group, acetyl or acyl group is directly linked either on the epsilon-amino group of said linker and wherein:

Xaa1 is arginine (R) or D-isomer arginine (R_{D});

Xaa2 is glutamine (Q) or D-isomer glutamine (Q_{D})

Xaa3 is leucine (L) or D-isomer leucine (L_{D});

Xaa4 is histidine (H) or α-aminoisobutyric acid (Aib);

Xaa5 is alanine (A) or D-isomer alanine (A_{D}) or glycine (G);

Xaa6 is methionine (M) or norleucine (Nle);

Xaa7 is phenylalanine (F) of 4-Br phenylalanine (4-BrF).

According to a particular embodiment of the present invention, the peptide analog of the present invention has a peptide covalently linked to the fluorocarbon group that is no more than 13 amino acid residues.

As used herein, the expression “completely hydrosoluble at physiological pH” means that the fluoropeptides of the present invention as above described have at least 50% hydrophobic amino acid residues and have an overall positive net charges and a final solubility in aqueous mixture above 100 μM by visual inspection of the cloudiness of the resulting dispersion and/or solubility measurements by classical physicochemical methods.

As used herein, the expression “non-immunogen” means that the fluoropeptides of the present invention as above described are not derived from any antigens capable of inducing immune response in an animal, including humans. Antigens may be derived from a virus, bacterium or mycobacterium, parasite, fungus, or any infectious agent or an autologous antigen or allergen. The fluoropeptides described in the invention are not included in any vaccine.

As used herein, the expression “metabolically stable” means that the fluoropeptides of the present invention as above described, have at least the half-life of the native peptide, in particular have a half-life at least twice longer than native peptide, of at least 20 min or more than one hour, as measured in the stability assay performed in human plasma.

As used herein, the expression “fluorocarbon group” includes either, perfluorocarbon (where all hydrogen are replaced by fluorine) or, hydrofluorocarbon (which contains both C—H and C—F bonds).

The fluorocarbon group may comprise one or more chains derived from perfluorocarbon or mixed fluorocarbon/hydrocarbon radicals, and may be saturated or unsaturated, each chain having from 3 to 30 carbon atoms. The fluorocarbon group is linked to the peptide through a covalent linkage, for example via NH₂-group of a lysine of the peptide of formula (I). The coupling to the peptide may be achieved through a functional group for linkage to —NH₂, being naturally present on the lysine of the peptide of formula I, or onto a linker. Modify the nature of the linkage between the fluorocarbon chain and the peptide allows to modulate the stability and/or solubility of the peptide. Examples of such linkages between the fluorocarbon chain and the peptide of formula I include amide, hydrazine, disulphide, thioether, ester, triazole and oxime bonds.

Optionally, a cleavable linker element (peptide or non-peptidic) may be incorporated to permit cleavage of the peptide from the fluorocarbon group. The linker may also be incorporated to assist in the synthesis of the fluoropeptide and to improve its stability and/or solubility, for example by including additional charges. So charged linker may be particularly useful especially if the peptide to which it is linked has no cationic aminoacids (i.e. lysine, histidine, arginine) at its N-terminal end. Examples of linkers include polyethylene glycol (PEG), or a peptide having about 1 to 6 amino acids, natural on non-natural ones, that may be cleaved by proteolytic enzymes or not. Preferably said amino acids are chosen from the group consisting of basic or aliphatic amino acids, more preferably from histidine, lysine, arginine and glycine. For example, the linker may be Arg-Gly-Arg.

Thus, the fluorocarbon group of the peptide analog of the present invention has chemical formula (II) CₘFₙ—C_{y}Hₓ(L) wherein m=3 to 30, n≤2m+1, y=0 to 2, x≤2y, (m+y)=3 to 30, and L, which is optional, is a functional group leading to covalent attachment to the peptide. For example said functional group is a carbonyle —C(O)— that forms an amide bond with the —NH2 of a lysine.

According to a particular embodiment of the above formula II of the fluorocarbon, m=5 to 15, preferably m=5 to 15 and y=1 to 4. According to another particular embodiment, the formula of the fluorocarbon group is perfluoroundecanoic acid of formula (A)

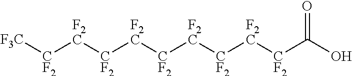 [SMILES: O=C(O)C(F)(F)C(F)(F)C(F)(F)C(F)(F)C(F)(F)C(F)(F)C(F)(F)C(F)(F)C(F)(F)C(F)(F)F]

or alternatively is 2H,2H,2H,3H,3H-perfluoroundecanoic acid of formula (B)

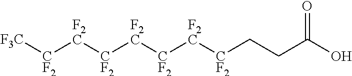 [SMILES: O=C(O)CCC(F)(F)C(F)(F)C(F)(F)C(F)(F)C(F)(F)C(F)(F)C(F)(F)C(F)(F)F]

or alternatively is heptadecafluoro-pentadecanoic acid of formula (C)

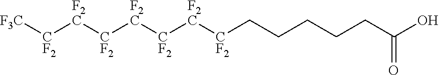 [SMILES: O=C(O)CCCCCC(F)(F)C(F)(F)C(F)(F)C(F)(F)C(F)(F)C(F)(F)C(F)(F)C(F)(F)F]

In these cases it is to be noted that reducing the length of the fluorocarbon group of formula (II), for example by deleting at least two CF₂ groups, preferably at least four CF₂ groups, can increase the solubility and/or plasmatic stability of the peptide to which it is linked.

According to a particular embodiment, the peptide analog of the present invention as above defined is further covalently linked to an acetyl group and/or an acyl group —C(O)R where R is a C₇₋₃₀ alkyl. For example it has the following formula (III) CH₃—C_{y}Hₓ—C(O)— wherein y=7 to 30, preferably y=10 to 20, more preferably y=14, and x=2y.

Said fluorocarbon group or further acetyl and/or acyl group can be linked at the N-terminal part of the peptide directly through a lysine, either on the alpha-amino or the epsilon-amino groups.

In one aspect, the peptide analog of the present invention includes but are not limited to neuropeptides or peptide hormones, a peptide mimetics or a fragment derived therefrom, for example Adiponectin, Adrenomedullin, Angiotensinogen, Angiotensin I, Angiotensin II, Angiopoietin-like Protein 8/Betatrophin, Amylin, Apelin, Apela Asprosin, Atrial Natriuretic Peptide/ANPO BNP, Betatrophin, Bradykinin, Calcitonin, Cholecystokinin, Chorionic Gonadotropin alpha Chain (HCG alpha), Chorionic Gonadotropin alpha/beta (HCG), CILP-1, Claudin-4, CNP, Copeptin, CRHBP, Corticotropin-releasing hormone, Dopamine, Enkephalin, Endothelin, Endothelin-1, Endothelin-2, Endothelin-3, Erythropoietin, FSH alpha/beta, FSH beta, Gastrin I, Gastrin-releasing Peptide/Bombesin, Gastric inhibitory polypeptide (GIP), Ghrelin/Obestatin, Growth hormone-releasing hormone (GHRH)Glucagon, GLP1, GLP2, Gonadotropin-releasing hormone (GnRH)GOAT/MBOAT4, Growth Hormone, Growth Hormone 2, Hepcidin, IGF-I, IGF-II, IGFBP-1, IGFBP-2, IGFBP-3, IGFBP-4, IGFBP-5, IGFBP-6, IGFBP-L1, IGFBP-rp1/IGFBP-7, IGFL-3, Incretins, INSL3, INSL4, INSL6, Insulin, Parathyroid hormone (PTH), Proinsulin, Irisin/FNDC5, Leptin/OB, LH alpha/beta Heterodimer, LH beta, Mas, MCT8/SLC16A2, Melanocyte-stimulating hormone, melatonin, Metastin/KiSS1, Motilin, Neuropeptide S/NPS, Neuropeptide Y/NPY, Neurophysin II, Obestatin, Orexin A/Hypocretin-1, Orexin B/Hypocretin-2, Osteocalcin, Oxytocin, PACAP/ADCYAP1, Pancreatic Polypeptide/PP, PITX2, Peptide YY (PYY), Placental Lactogen/CSH1, Prolactin, Proliferin, Proliferin-related Protein/Plfr, Proprotein Convertase 2/PCSK2, PSG-1, PSG-6, PTH, PTHLH/PTHrP, Retinol Binding Protein 4, Relaxin-1, Relaxin-2, Relaxin-3, Renalase, Secretin, Serpin A8/Angiotensinogen, SOCS-2, Somatostatin, Stanniocalcin 1/STC-1, Stanniocalcin 2/STC-2, Substance P, Thrombopoietin, Thyroglobulin, Thyroxine, Transthyretin/Prealbumin Thyrotropin-releasing hormone (TRH), TSH alpha/beta Heterodimer, TSH beta, Urocortin, Urotensin-II, V1b Vasopressin/Arg8-Vasopressin, VIP.

In one aspect, the peptide analog of the present invention includes but are not limited to natural or non-natural peptide which is capable of interacting with AGTR-1, AGTR-2, Bradykinin RB1/BDKRB1, BRS3, C5L2/GPR77, Calcitonin R, CCK-A R, Cholecystokinin-B R/CCKBR, Pro-Corticoliberin/Pro-CRH, CRHR1, CRHR2, CRLR, EDNRA/Endothelin R Type A, ELTD1, Endothelin, EDNRB/Endothelin R Type B, Erythropoietin R, FSH R, Gastrin-releasing Peptide R/GRPR, GHRHR, GHSR, GIPR, GLP-1R, GLP-2R, Glucagon R/GCGR, GnRHR, GPR10, GPR39, Growth Hormone R/GHR, IGF-I R, IGFLR1, Insulin R/CD220, Insulin R/IGF-I R Heterotetramer, INSRR, IRS1, IRS2, KiSS1R/GPR54, Leptin R, Lgr6, LHR, MCHR1, MCHR2 Melanocortin-1 R/MC1R, Melanocortin-2 R/MC2R, Melanocortin-3 R/MC3R, Melanocortin-4 R, Melanocortin-5 R/MC5R, Motilin R/GPR38, NK1R, NK2R, NK3R/TACR3, NPR-1, Orexin R1/HCRTR1, Orexin R2/HCRTR2, OXTR, Prokineticin R1/PROKR1, Prokineticin R2/PROKR2, Prolactin R, PTH1R/PTHR1, PTH2R, RAMP1, RAMP2, RAMP3, Relaxin R1, Relaxin R2, RXFP3/RLN3R1, RXFP4/GPCR142, Secretin R, Somatostatin R1/SSTR1, Somatostatin R3/SSTR3, Somatostatin R4/SSTR4, Somatostatin R2/SSTR2, Somatostatin R5/SSTR5, TRHR, TSH R, Urotensin-II R, Vasopressin R/AVPR1B, V2 Vasopressin R/AVPR2, V1a Vasopressin R/AVPR1A, VIP R1/VPAC1

According to a particular embodiment, the peptide analog of the present invention is selected from:
i) an apelin analog having said peptide of the formula (IV) (SEQ ID NO: 2):

Xaa1-Arg-Pro-Xaa2-Leu-Xaa3-Xaa4-Xaa5-Gly-Pro-Xaa6-Pro-Xaa7 (IV)

and wherein

Xaa1 is L- or D-glutamine (QL or QD) or alanine (A);

Xaa2 is arginine (R) or lysine (K) or D-norleucine (Nle);

Xaa3 is serine (S) or alanine (A);

Xaa4 is histidine (H) or alanine (A);

Xaa5 is lysine (K) or norleucine (Nle);

Xaa6 is methionine (M), leucine (L), phenylalanine (F) or norleucine (Nle);

Xaa7 is phenylalanine (F), 4-Br phenylalanine (4-BrF), 4-(Obenzyl)phenylalanine (4-ObnF) or p-benzoyl phenylalanine (Bpa); and
ii) an apelin analog with an amino acid sequence having at least 80% identity with the sequence of (i).

According to another particular embodiment, a peptide analog of the present invention is selected from the group consisting of:

i) an angiotensin II analog having said peptide of the formula (V) (SEQ ID NO: 3):

Xaa1-Arg-Val-Tyr-Ile-His-Pro-Xaa2 (V)

and wherein

Xaa1 is aspartic acid (D) or sarcosine;

Xaa2 is phenylalanine or OH; and

ii) an angiotensin II analog with an amino acid sequence having at least 80% identity with the sequence of (i).

According to another particular embodiment, a peptide analog of the present invention is selected from the group consisting of:

i) an oxytocin analog having said peptide of the formula (VI) (SEQ ID NO: 4):

Cys-Tyr-Ile-Xaa1-Asp-Cys-Xaa2-Xaa3-Gly (VI)

and wherein

Xaa1 is glutamine or threonine;

Xaa2 is proline or glycine;

Xaa3 is leucine, lysine or proline;

ii) an oxytocin analog with an amino acid sequence having at least 80% identity with the sequence of (i).

In another aspect, the present invention also relates to a peptide analog of the present invention, for use as a drug.

In another aspect, the present invention also relates to a peptide analog of the present invention, for use in the prevention and/or treatment of a GPCR-related disease or GPCR-related disorder, preferably selected from central nervous system (CNS) disorders, cardiovascular disorders, nociception, renal disorders, neuroinflammation, etc . . . . For example said diseases or disorders include, but are not limited to:
cardiovascular disease: heart failure, kidney diseases (e.g. renal failure, nephritis, etc. . . . ), hypertension, pulmonary hypertension, cirrhosis, arteriosclerosis, pulmonary emphysema, pulmonary oedema, stroke, brain ischemia, myocardial impairment in sepsis, card iomyopathy;
the syndrome of inappropriate antidiuretic hormone (SIADH);
metabolic diseases: obesity, anorexia, hyperphagia, polyphagia, hypercholesterolemia, hyperglyceridemia, hyperlipemia;
various types of dementia: senile dementia, Alzheimer's disease, cerebrovascular dementia, dementia due to genealogical denaturation degenerative disesases, dementia resulting from infectious diseases, dementia associated with endocrine diseases, metabolic diseases, or poisoning, dementia caused by tumors, and dementia due to traumatic diseases, depression, hyperactive child syndrome, disturbance of consciousness, anxiety disorder, schizophrenia, phobia;
pain and hyperalgesia.

For example said therapeutic peptides of the present invention include, but are not limited to the following peptides:

In cardiovascular disease:

| | |
|---|---|
| Balixafortide | ACSAPRYCYQKPPYH (SEQ ID NO: 5) |
| BIS-5409/MR-409 | YADAIFTNSYRKVLGQLSARKLLQDIMSR (SEQ ID NO: 6) |
| Bivalirudin | FPRPGGGGNGDFEEIPEEYL (SEQ ID NO: 7) |
| BQ-123 | DPVLY (SEQ ID NO: 8) |
| Carperitide | SLRRSSCFGGRMDRIGAQSGLGCNSFRY (SEQ ID NO: 9) |
| CEL-1000 | DGQEEKAGVVSTGLIGGG (SEQ ID NO: 10) |
| Cenderitide | GLSKGCFGLKLDRIGSMSGLGCPSLRDPRPNAPSTSA (SEQ ID |
| | NO: 11) |
| CGEN-8565 | FLGYCIYLNKRRGDPAFKRRLRD (SEQ ID NO: 12) |
| Des-Asp | DRVYIHPFHL ((SEQ ID NO: 13) |
| Angiotensin 1 | |
| Desirudin | VVYTDCTESGQNLCLCEGSNVCGQGNKCILGSDGEKNQCVTGE |
| | GTPKPQSHNDGDFEEIPEEYLQ (SEQ ID NO: 14) |
| Elamipretide | R-2,6-dimethyl-YKF (SEQ ID NO: 15) |
| FX-06 | RGHRPLDKKREEAPSLRPAPPPISGGGY (SEQ ID NO: 16) |
| LJPC-501 | DRVYIHPF (SEQ ID NO: 17) |
| (Angiotensin II) | |
| MP-3167 | AVSNADLMDFKNLLDHLEEKMPLEDEVVPPQVLSERN (SEQ ID |
| (Vastiras) | NO: 18) |
| NA-1 | YRKKRRQRRRKLSSIESDV (SEQ ID NO: 19) |
| NBI-69734 | IVLSLDVPIGLLQILLEQARARAAREQATTNARILARVGH (SEQ ID |
| (Urocortin II) | NO: 20) |
| Nesiritide | SPKMVQGSGCFGRKMDRISSSSGLGCKVLRRH (SEQ ID NO: 21) |
| PL-3994 | CHFAGRXDRISCYR; X = Nle (SEQ ID NO: 22) |
| RGN-352 | MSDKPDMAEIEKFDKSKLKKTETQEKNPLPSKETIEQEKQAGES |
| (Thymosin beta-4) | (SEQ ID NO: 23) |
| Selepressin | (2S)-1-[(4R,7S,10S,13S,16S,19R)-19-amino-10-(4-amino-4- |
| | oxobutyl)-7-(2-amino-2-oxoethyl)-16-benzyl-13-[(2S)-butan-2-yl]- |
| | 6,9,12,15,18-pentaoxo-1,2-dithia-5,8,11,14,17- |
| | pentazacycloicosane-4-carbonyl]-N-[(2S)-1-[(2-amino-2- |
| | oxoethyl)amino]-1-oxo-5-(propan-2-ylamino)pentan-2-yl]pyrrolidine- |
| | 2-carboxamide |
| Solnatide | CGQRETPEGAEAKPWYC (SEQ ID NO: 24) |
| Terlipressin | (2S)-1-[(4R,7S,10S,13S,16S,19R)-19-[[2-[[2-[(2- |
| | aminoacetyl)amino]acetyl]amino]acetyl]amino]-7-(2-amino-2- |
| | oxoethyl)-10-(3-amino-3-oxopropyl)-13-benzyl-16-[(4- |
| | hydroxyphenyl)methyl]-6,9,12,15,18-pentaoxo-1,2-dithia- |
| | 5,8,11,14,17-pentazacycloicosane-4-carbonyl]-N-[(2S)-6-amino-1- |
| | [(2-amino-2-oxoethyl)amino]-1-oxohexan-2-yl]pyrrolidine-2- |
| | carboxamide |
| THR-18 | (4S)-4-[[(2S)-1-[(2S)-2-[[(2S)-2-[[(2S)-2-acetamido-5- |
| | carbamimidamidopentanoyl]amino]-4- |
| | methylsulfanylbutanoyl]amino]propanoyl]pyrrolidine-2- |
| | carbonyl]amino]-5-[[(2S)-1-[[(2S,3S)-1-[[(2S,3S)-1-[[(2S)-1-[[(2S)-1- |
| | [[(2S)-1-[(2S)-2-[[1-[[(2S)-1-[[(2S)-1-[[(2S)-1-[[1-[[(2S)-1-amino-5- |
| | carbamimidamido-1-oxopentan-2-yl]amino]-3-methyl-1-oxobutan-2- |
| | yl]amino]-3-methyl-1-oxobutan-2-yl]amino]-3-(4-hydroxyphenyl)-1- |
| | oxopropan-2-yl]amino]-4-methyl-1-oxopentan-2-yl]amino]-1-oxo-3- |
| | phenylpropan-2-yl]carbamoyl]pyrrolidin-1-yl]-5-carbamimidamido-1- |
| | oxopentan-2-yl]amino]-3-carboxy-1-oxopropan-2-yl]amino]-4- |
| | methylsulfanyl-1-oxobutan-2-yl]amino]-3-methyl-1-oxopentan-2- |
| | yl]amino]-3-methyl-1-oxopentan-2-yl]amino]-4-carboxy-1-oxobutan- |
| | 2-yl]amino]-5-oxopentanoic acid; 2,2,2-trifluoroacetic acid |
| TRV027 | GRVYIHPA (SEQ ID NO: 25) |
| Ularitide | TAPRSLRRSSCFGGRMDRIGAQSGLGCNSFRY (SEQ ID NO: 26) |
| (Urodilatin) | |

In various central nervous system diseases:

| | |
|---|---|
| acALY-18 | ALYDKGYTSKEQKDCVG (SEQ ID NO: 27) |
| Cibinetide | 5-Oxo-PEQLERALDSS (SEQ ID NO: 28) |
| COG-112 | RQIKIWFQNRRMKWKKC (SEQ ID NO: 29) |
| corticotropin | SYSMEHFRWGKPVGKKRRPVKVYPDGAEDQLAEAFPLEF (SEQ ID |
| | NO: 30) |
| CR-845 | 4-Piperidinecarboxylic acid, 4-amino-1-[(2R)-6-amino-2-[[(2R)-2-[[(2R)- |
| (Difelikefalin ) | 2-[[(2R)-2-amino-1-oxo-3-phenylpropyl]amino]-1-oxo-3- |
| | phenylpropyl]amino]-4-methyl-1-oxopentyl]amino]-1-oxohexyl] |
| davunetide | (2S)-5-amino-2-[[(2S)-1-[(2S,3S)-2-[[(2S)-2-[[(2S)-2-[[(2S)-1-[(2S)-2- |
| | [[(2S)-2,4-diamino-4-oxobutanoyl]amino]propanoyl]pyrrolidine-2- |
| | carbonyl]amino]-3-methylbutanoyl]amino]-3-hydroxypropanoyl]amino]- |
| | 3-methylpentanoyl]pyrrolidine-2-carbonyl]amino]-5-oxopentanoic acid |
| elcatonin | 1,7-Dicarbacalcitonin (salmon), 1-butanoic acid-26-L-aspartic acid-27- |
| | L-valine-29-L-alanine- |
| glatiramer | acetic acid; 2-amino-3-(4-hydroxyphenyl)propanoic acid; 2- |
| acetate | aminopentanedioic acid; 2-aminopropanoic acid; 2,6-diaminohexanoic |
| | acid |
| leuprorelin ER | N-[1-[[1-[[1-[[1-[[1-[[1-[[5-(diaminomethylideneamino)-1-[2- |
| | (ethylcarbamoyl)pyrrolidin-1-yl]-1-oxopentan-2-yl]amino]-4-methyl-1- |
| | oxopentan-2-yl]amino]-4-methyl-1-oxopentan-2-yl]amino]-3-(4- |
| | hydroxyphenyl)-1-oxopropan-2-yl]amino]-3-hydroxy-1-oxopropan-2- |
| | yl]amino]-3-(1H-indol-3-yl)-1-oxopropan-2-yl]amino]-3-(1H-imidazol-5- |
| | yl)-1-oxopropan-2-yl]-5-oxopyrrolidine-2-carboxamide |
| PMZ-2123 | DPVLW (SEQ ID NO: 31) |
| PT-320 | SHGEGTFATSDLSKQMQQQAVRLFIQWLKDGGPSSGAPPP (SEQ |
| | ID NO: 32) |
| Relamorelin | 4-[[(2S)-2-[[(2R)-2-[[(2R)-3-(1-benzothiophen-3-yl)-2-(piperidine-3- |
| | carbonylamino)propanoyl]amino]-3-(1H-indo1-3-yl)propanoyl]amino]-3- |
| | phenylpropanoyl]amino]pipendine-4-carboxamide |
| Ziconotide | CKGKGAKCSRLMYDCCTGSCRSGKC (SEQ ID NO: 33) |

In various Dermatologic diseases:

| | |
|---|---|
| AB-103 | SPMLVAYD (SEQ ID NO: 34) |
| AG-30/5C | MLKLIFLHRLKRMRKRLKRKLRLWHRKRYK (SEQ ID NO: 35) |
| Bacitracin | (4R)-5-[[1-[[(3R,9R,15R)-15-(3-aminopropyl)-9-benzyl-12-butan-2-yl-3- |
| | (carboxymethyl)-6-(1H-imidazol-5-ylmethyl)-2,5,8,11,14,17-hexaoxo- |
| | 1,4,7,10,13,16-hexazacyclodocos-18-yl]amino]-3-methyl-1-oxopentan- |
| | 2-yl]amino]-4-[[4-methyl-2-[[2-(3-methylpentan-2-yl)-4,5-dihydro-1,3- |
| | thiazole-4-carbonyl]amino]pentanoyl]amino]-5-oxopentanoic acid |
| Desloratadine | 8-chloro-11-piperidin-4-ylidene-5,6-dihydrobenzo[1,2]cyclohepta[2,4- |
| | b]pyridine |
| FOL-005 | VDTYDGDISWYGLR (SEQ ID NO: 67) |
| Granexin | RQPKIWFPNRRKPWKKRPRPDDLEI (SEQ ID NO: 37) |
| LL-37 | LLGDFFRKSKEKIGKEFKRIVQRIKDFLRNLVPRTES (SEQ ID NO: 38) |
| Omiganan | ILRWPWWPWRRK (SEQ ID NO: 39) |
| PXL-01 | EATKCFQWQRNMRKVRGPPVSCIKR (SEQ ID NO: 40) |
| RGN-137 | MSDKPDMAEIEKFDKSKLKKTETQEKNPLPSKETIEQEKQAGES (SEQ ID NO: 41) |
| SR-0379 | MLKLIFLHRLKRMRKRLKRK (SEQ ID NO: 42) |

In various ear nose throat disorders:

| | |
|---|---|
| B-2088 | RGRKVVRRKKRRVVKRGR |
| | (SEQ ID NO: 43) |
| XG-102 | DQSRPVQPFLNLTTPRKPRPPRRRQRRKKRG |
| | (SEQ ID NO: 44) |

In various aastrointestinal diseases:

| | |
|---|---|
| Colistin Sulfate | N-[(2S)-4-amino-1-[[(2S,3R)-1-[[(2S)-4-amino-1-oxo-1- |
| | [[(3S,6S,9S,12S,15R,18S,21S)-6,9,18-tris(2-aminoethyl)-3-[(1R)-1- |
| | hydroxyethyl]-12,15-bis(2-methylpropyl)-2,5,8,11,14,17,20-heptaoxo- |
| | 1,4,7,10,13,16,19-heptazacyclotricos-21-yl]amino]butan-2-yl]amino]-3- |
| | hydroxy-1-oxobutan-2-yl]amino]-1-oxobutan-2-yl]-6- |
| | methyloctanamide; sulfuric acid |
| Cosyntropin ER | SYSMEHFRWGKPVGKKRRPVKVYP (SEQ ID NO: 45) |
| FE-203799 | HGDGSFSDEMFTILDLLAARDFFINWLIQTKITD (SEQ ID NO: 46) |
| Linaclotide | CCEYCCNPACTGCY (SEQ ID NO: 47) |
| NB-1001 | GLP-1-XTEN |
| Octreotide | FCFWKXCX X: DL-Threonine (SEQ ID NO: 48) |
| Octreotide | FCFWKTCT (SEQ ID NO: 49) |
| Acetate | |
| Plecanatide | NDECELCVNVACTGCL (SEQ ID NO: 50) |
| Recanaclotide | CCXLCCNPACTGC X: Ser(PO3H2) (SEQ ID NO: 51) |
| Somatostatin | AGCKNFFWKXFXSC X: DL-Threonine (SEQ ID NO: 52) |
| Teduglutide | HGDGSFSDEMNTILDNLAARDFINWLIQTKITD (SEQ ID NO: 53) |
| Vapreotide | acetic acid; 10-(4-aminobutyl)-N-[1-amino-3-(1H-indol-2-yl)-1- |
| Acetate | oxopropan-2-yl]-19-[(2-amino-3-phenylpropanoyl)amino]-16-[(4- |
| | hydroxyphenyl)methyl]-13-(1H-indol-3-ylmethyl)-6,9,12,15,18- |
| | pentaoxo-7-propan-2-yl-1,2-dithia-5,8,11,14,17-pentazacycloicosane- |
| | 4-carboxamide |
| Vasopressin | N-[6-amino-1-[(2-amino-2-oxoethyl)amino]-1-oxohexan-2-yl]-1-[19- |
| | amino-7-(2-amino-2-oxoethyl)-10-(3-amino-3-oxopropyl)-13-benzyl-16- |
| | [(4-hydroxyphenyl)methyl]-6,9,12,15,18-pentaoxo-1,2-dithia- |
| | 5,8,11,14,17-pentazacycloicosane-4-carbonyl]pyrrolidine-2- |
| | carboxamide; 1-[19-amino-7-(2-amino-2-oxoethyl)-10-(3-amino-3- |
| | oxopropyl)-13-benzyl-16-[(4-hydroxyphenyl)methyl]-6,9,12,15,18- |
| | pentaoxo-1,2-dithia-5,8,11,14,17-pentazacycloicosane-4-carbonyl]-N- |
| | [1-[(2-amino-2-oxoethyl)amino]-5-(diaminomethylideneamino)-1- |
| | oxopentan-2-yl]pyrrolidine-2-carboxamide |
| ZP-1848 | HGEGTFSSELATILDALAARDFIAWLIATKITD (SEQ ID NO: 54) |

In various aenetic disorders diseases:

| | |
|---|---|
| Afamelanotide | SYSXEHFRWGKPV; X: Nle (SEQ ID NO: 55) |
| Alisporivir | (3S,6S,9S,12R,15S,18S,21S,24S,27R,30S,33S)-25,30-diethyl-33- |
| | [(E,1R,2R)-1-hydroxy-2-methylhex-4-enyl]-1,4,7,10,12,15,19,27,28- |
| | nonamethyl-6,9,18-tris(2-methylpropyl)-3,21,24-tri(propan-2-yl)- |
| | 1,4,7,10,13,16,19,22,25,28,31-undecazacyclotritriacontane- |
| | 2,5,8,11,14,17,20,23,26,29,32-undecone |
| AZP-531 | SPEHQRVQ (SEQ ID NO: 56) |
| LJPC-401 | DTHFPICIFCCGCCHRSKCGMCCKT (SEQ ID NO: 57) |
| Setmelanotide | RCAHFRWC (SEQ ID NO: 58) |
| TXA-127 | DRVYIHP (SEQ ID NO: 59) |

In various genito-urinary system and sexual hormones disorders:

| | |
|---|---|
| AMY-101 | YICVWQDWXHRCI; X: Sar (SEQ ID NO: 60) |
| Cardiotoxin | LKCNKLVPLFYKTCPAGKNLCYKMFMVSNKMVPVKRGCIDVCPKSSLLVKYVCCNTDRCN (SEQ |
| | ID NO: 61) |
| Desmopressin | (2S)-N-[(2R)-1-[(2-amino-2-oxoethyl)amino]-5- |
| | (diaminomethylideneamino)-1-oxopentan-2-yl]-1- |
| | [(4R,7S,10S,13S,16S)-7-(2-amino-2-oxoethyl)-10-(3-amino-3- |
| | oxopropyl)-13-benzyl-16-[(4-hydroxyphenyl)methyl]-6,9,12,15,18- |
| | pentaoxo-1,2-dithia-5,8,11,14,17-pentazacycloicosane-4- |
| | carbonyl]pyrrolidine-2-carboxamide |
| Lypressin | N-[6-amino-1-[(2-amino-2-oxoethyl)amino]-1-oxohexan-2-yl]-1-[19- |
| | amino-7-(2-amino-2-oxoethyl)-10-(3-amino-3-oxopropyl)-13-benzyl-16- |
| | [(4-hydroxyphenyl)methyl]-6,9,12,15,18-pentaoxo-1,2-dithia- |
| | 5,8,11,14,17-pentazacycloicosane-4-carbonyl]pyrrolidine-2- |
| | carboxamide |

In various hematological disorders:

| | |
|---|---|
| Aprotinin | RPDFCLEPPYTGPCRARIIRYFYNAKAGLCQTFVVGGCRAKRNNFKSAEDCERTCGGA (SEQ ID |
| | NO: 62) |
| BL-8040 | RRX₁CYXKKPYRXCR; X₁: NaI, X: Cit (SEQ ID NO: 63) |
| CBLB-612 | VQGEESNDK (SEQ ID NO: 64) |
| EPO-018B | GGTYSCHFGALTWVCRPQRGA (SEQ ID NO: 65) |
| Peginesatide | Poly(oxy-1,2-ethanediyl), alpha-hydro-omega-methoxy-, diester with |
| | 21N6,21′N6-[[(N2,N6-dicarboxylysyl-beta-alanyl)imino]bis(1-oxo-2,1- |
| | ethanediyl)]bis[N-acetylglycylglycyl-L-leucyl-L-tyrosyl-L-alanyl-L- |
| | cysteinyl-L-histidyl-L-methionylglycyl-L-prolyl-L-isoleucyl-L-threonyl- |
| | 3-(1-naphthalenyl)-L-alanyl-L-valyl-L-cysteinyl-L-glutaminyl-L-prolyl- |
| | L-leucyl-L-arginyl-N-methylglycyl-L-lysinamide]cyclic(6-15),(6′-15′)- |
| | bis(disulfide) |

In various hormonal disorders:

| | |
|---|---|
| COR-005 | cyclo(-y-aminobutyryl-L-phenylalanyl-L-tryptophanyl-D-tryptophanyl-L- |
| | lysyl-L-threonly-L-phenylalanyl-N-3-carboxypropyl)-glycine amide, |
| | acetate salt |
| Etelcalcetide | CARRRAR (SEQ ID NO: 66) |
| Hydrochloride | |
| Gonadorelin | acetic acid; (2S)-N-[(2S)-1-[[(2S)-1-[[(2S)-1-[[(2S)-1-[[2-[[(2S)-1- |
| Acetate | [[(2S)-1-[(2S)-2-[(2-amino-2-oxoethyl)carbamoyl]pyrrolidin-1-yl]-5- |
| | (diaminomethylideneamino)-1-oxopentan-2-yl]amino]-4-methyl-1- |
| | oxopentan-2-yl]amino]-2-oxoethyl]amino]-1-oxo-3-phenylpropan-2- |
| | yl]amino]-3-hydroxy-1-oxopropan-2-yl]amino]-3-(1H-indol-3-yl)-1- |
| | oxopropan-2-yl]amino]-3-(1H-imidazol-5-yl)-1-oxopropan-2-yl]-5- |
| | oxopyrrolidine-2-carboxamide; hydrate |
| histrelin | (2S)-N-[(2S)-1-[[(2S)-1-[[(2S)-1-[[(2S)-1-[[(2R)-3-(1-benzylimidazol-4- |
| | yl)-1-[[(2S)-1-[[(2S)-5-(diaminomethylideneamino)-1-[(2S)-2- |
| | (ethylcarbamoyl)pyrrolidin-1-yl]-1-oxopentan-2-yl]amino]-4-methyl-1- |
| | oxopentan-2-yl]amino]-1-oxopropan-2-yl]amino]-3-(4-hydroxyphenyl)- |
| | 1-oxopropan-2-yl]amino]-3-hydroxy-1-oxopropan-2-yl]amino]-3-(1H- |
| | indol-3-yl)-1-oxopropan-2-yl]amino]-3-(1H-imidazol-5-yl)-1-oxopropan- |
| | 2-yl]-5-oxopyrrolidine-2-carboxamide |
| Lanreotide | acetic acid; (4R,7S,10S,13R,16S,19R)-10-(4-aminobutyl)-N-[(2S,3R)-1- |
| Acetate | amino-3-hydroxy-1-oxobutan-2-yl]-19-[[(2R)-2-amino-3-naphthalen-2- |
| | ylpropanoyl]amino]-16-[(4-hydroxyphenyl)methyl]-13-(1H-indol-3- |
| | ylmethyl)-6,9,12,15,18-pentaoxo-7-propan-2-yl-1,2-dithia-5,8,11,14,17- |
| | pentazacycloicosane-4-carboxamide |
| MOD-4023 | MATGSRTSLLLAFGLLCLPWLQEGSASSSSKAPPPSLPSPSRLPGPS |
| | DTPILPQFPTIPLSRLFDNAMLRAHRLHQLAFDTYQEFEEAYIPKEQK |
| | YSFLQNPQTSLCFSESIPTPSNREETQQKSNLELLRISLLLIQSWLEPV |
| | QFLRSVFANSLVYGASDSNVYDLLKDLEEGIQTLMGRLEDGSPRTG |
| | QIFKQTYSKFDTNSHNDDALLKNYGLLYCFRKDMDKVETFLRIVQCR |
| | SVEGSCGFSSSSKAPPPSLPSPSRLPGPSDTPILPQSSSSKAPPPSL |
| | PSPSRLPGPSDTPILPQ (SEQ ID NO: 67) |
| Octreotide | acetic acid; 10-(4-aminobutyl)-19-[(2-amino-3-phenylpropanoyl)amino]- |
| Acetate Long | 16-benzyl-N-(1,3-dihydroxybutan-2-yl)-7-(1-hydroxyethyl)-13-(1H-indol- |
| Acting | 3-ylmethyl)-6,9,12,15,18-pentaoxo-1,2-dithia-5,8,11,14,17- |
| | pentazacycloicosane-4-carboxamide |
| Pasireotide ER | [(20R)-9-(4-aminobutyl)-3-benzyl-12-(1H-indol-3-ylmethyl)- |
| | 2,5,8,11,14,17-hexaoxo-15-phenyl-6-[(4- |
| | phenylmethoxyphenyl)methyl]-1,4,7,10,13,16- |
| | hexazabicyclo[16.3.0]henicosan-20-yl]N-(2-aminoethyl)carbamate |
| Somatrem | Somatotropin (human) |
| Triptorelin | N-[1-[[1-[[1-[[1-[[1-[[1-[[1-[2-[(2-amino-2-oxoethyl)carbamoyl]pyrrolidin- |
| | 1-yl]-5-(diaminomethylideneamino)-1-oxopentan-2-yl]amino]-4-methyl- |
| | 1-oxopentan-2-yl]amino]-3-(1H-indol-3-yl)-1-oxopropan-2-yl]amino]-3- |
| | (4-hydroxyphenyl)-1-oxopropan-2-yl]amino]-3-hydroxy-1-oxopropan-2- |
| | yl]amino]-3-(1H-indol-3-yl)-1-oxopropan-2-yl]amino]-3-(1H-imidazol-5- |
| | yl)-1-oxopropan-2-yl]-5-oxopyrrolidine-2-carboxamide |
| Triptorelin | (2S)-N-[(2S)-1-[[(2S)-1-[[(2S)-1-[[(2S)-1-[[(2R)-1-[[(2S)-1-[[(2S)-1-[(2S)- |
| Pamoate | 2-[(2-amino-2-oxoethyl)carbamoyl]pyrrolidin-1-yl]-5- |
| | (diaminomethylideneamino)-1-oxopentan-2-yl]amino]-4-methyl-1- |
| | oxopentan-2-yl]amino]-3-(1H-indol-3-yl)-1-oxopropan-2-yl]amino]-3-(4- |
| | hydroxyphenyl)-1-oxopropan-2-yl]amino]-3-hydroxy-1-oxopropan-2- |
| | yl]amino]-3-(1H-indol-3-yl)-1-oxopropan-2-yl]amino]-3-(1H-imidazol-5- |
| | yl)-1-oxopropan-2-yl]-5-oxopyrrolidine-2-carboxamide; 4-[(3-carboxy-2- |
| | hydroxynaphthalen-1-yl)methyl]-3-hydroxynaphthalene-2-carboxylic |
| | acid |

In various immunological disorders:

| | |
|---|---|
| Aclerastide | 1-7-Angiotensin II, 3-L-norleucine-5-L-isoleucine |
| ATI-2341 | MGYQKKLRSMTDKYRL (SEQ ID NO: 68) |
| Dalazatide | o-PHOSPHONO-L-Tyrosyl-2-(2-(2- |
| | aminoethoxy)ethoxy)acetyl(potassium channel toxin kappa-stichotoxin- |
| | shela stoichactis helianthus (caribbean sea anemone)) peptidamide |
| Disitertide | (2S)-4-amino-2-[[(2S)-5-amino-2-[[(2S)-2-[[(2S)-2-[[(2S)-2-[[(2S)-2- |
| | [[(2S,3S)-2-[[(2S,3S)-2-[[(2S)-2-[[(2S)-2-[[(2S)-2-[[(2S)-2-[[(2S)-2- |
| | [[(2S,3R)-2-amino-3-ydroxybutanoyl]amino]-3- |
| | hydroxypropanoyl]amino]-4-methylpentanoyl]amino]-3- |
| | carboxypropanoyl]amino]propanoyl]amino]-3- |
| | hydroxypropanoyl]amino]-3-methylpentanoyl]amino]-3- |
| | thylpentanoyl]amino]-3-(1H-indol-3- |
| | yl)propanoyl]amino]propanoyl]amino]-4-methylsulfanylbutanoyl]amino]- |
| | 4-methylsulfanylbutanoyl]amino]-5-oxopentanoyl]amino]-4-oxobutanoic |
| | acid |
| Edratide | GYYWSWIRQPPGKGEEWIG (SEQ ID NO: 69) |
| Forigerimod | RIHMVYSKRXGKPRGYAFIEY, acetate X: DL-Threonine (SEQ ID NO: 70) |
| Acetate | |
| hPTH-1-37 | SVSEIQLMHNLGKHLNSMERVEWLRKKLQDVHNFVAL ((SEQ ID NO: 71) |
| Icatibant | (2S)-2-[[(3aS,7aS)-1-[(3R)-2-[(2S)-2-[[(2S)-2-[[2-[[(2S,4R)-1-[(2S)-1- |
| Acetate | [(2S)-2-[[(2R)-2-amino-5-(diaminomethylideneamino)pentanoyl]amino]- |
| | 5-(diaminomethylideneamino)pentanoyl]pyrrolidine-2-carbonyl]-4- |
| | hydroxypyrrolidine-2-carbonyl]amino]acetyl]amino]-3-thiophen-2- |
| | ylpropanoyl]amino]-3-hydroxypropanoyl]-3,4-dihydro-1H-isoquinoline- |
| | 3-carbonyl]-2,3,3a,4,5,6,7,7a-octahydroindole-2-carbonyl]amino]-5- |
| | (diaminomethylideneamino)pentanoic acid; acetic acid |

In various infectious diseases:

| | |
|---|---|
| ALB-408 | LVRYTKKVPQVSTPTL (SEQ ID NO: 72) |
| Enfuvirtide | YTSLIHSLIEESQNQQEKNEQELLELDKWASLWNWF (SEQ ID NO: 73) |
| FB-006M | WEEWDREINNYTKLIHELIEESQNQQEKNEQELL (SEQ ID NO: 74) |
| LTX-109 | (2S)-2-amino-5-(diaminomethylideneamino)-N-[(2S)-1-[[(2S)-5- |
| | (diaminomethylideneamino)-1-oxo-1-(2-phenylethylamino)pentan-2- |
| | yl]amino]-1-oxo-3-(2,5,7-tritert-butyl-1H-indol-3-yl)propan-2- |
| | yl]pentanamide |
| NP-213 | 2-[3-[(2S,5S,8S,11S,14S,17S,20S)-5,8,11,14,17,20-hexakis[3- |
| | (diaminomethylideneamino)propyl]-3,6,9,12,15,18,21-heptaoxo- |
| | 1,4,7,10,13,16,19-heptazacyclohenicos-2-yl]propyl]guanidine |
| Thymalfasin | SDAAVDTSSEITTKDLKEKKEVVEEAEN (SEQ ID NO: 75) |
| Pidotimod | (4R)-3-[(2S)-5-oxopyrrolidine-2-carbonyl]-1,3-thiazolidine-4- |
| | carboxylic acid |
| Sifuvirtide | SWETWEREIENYTRQIYRILEESQEQQDRNERDLLE (SEQ ID NO: 76) |
| Thymalfasin | SDAAVDTSSEITTKDLKEKKEVVEEAEN (SEQ ID NO: 77) |
| VIR-576 | LEAIPCSIPPEFLFGKPFVFLEAIPCSIPPEFLFGKPFVF (SEQ ID NO: 78) |

In various male health diseases:

| | |
|---|---|
| Chorionic | Gonadotrophin |
| Gonadotrophin | |
| TAK-448 | 2-(N-Acetyl-d-tyrosyl-trans-4- |
| | hydroxy-1-prolyl-1-asparaginyl-1- |
| | threonyl-1-phenylalanyl) |
| | hydrazinocarbonyl-1-leucyl-Nω- |
| | methyl-1-arginyl-1-tryptophanamide |
| | monoacetate |

In various metabolic disorders:

| | |
|---|---|
| abaloparatide | C2.29-methyl(22-L-glutamic acid(F>E),23-L-leucine(F>L),25-L- |
| | glutamic acid(H>E),26-L-lysine(H>K),28-L-leucine(I>L),30-L- |
| | lysine(E>K),31-L-leucine(I>L))human parathyroid hormone-related |
| | protein-(1-34)-proteinamide |
| AC-163794 | EGTFISDYSIAMDKIHQQDFVNWLLAQKPSSGAPPPS (SEQ ID NO: 79) |
| Albenatide | S3.34-{1-[(23S)-23-{[exendin-4 Heloderma suspectum precursor-(48- |
| | 86)-peptidyl (exenatidyl)amino}-3,12,24-trioxo-7,10-dioxa-4,13,18,25- |
| | tetraazapentacosyl]-2,5-dioxopyrrolidin-3-yl}human serum albumin |
| AMG-5041 | (2S)-2-[[(2S,3R)-2-[[2-[[(2S)-5-amino-2-[[(2S)-2-[[(2S)-2-amino-3-(1H- |
| | imidazol-5-yl)propanoyl]amino]-3-hydroxypropanoyl]amino]-5- |
| | oxopentanoyl]amino]acetyl]amino]-3-hydroxybutanoyl]amino]-3- |
| | phenylpropanoic acid |
| Argipressin | 1-[19-amino-7-(2-amino-2-oxoethyl)-10-(3-amino-3-oxopropyl)-13- |
| | benzyl-16-[(4-hydroxyphenyl)methyl]-6,9,12,15,18-pentaoxo-1,2-dithia- |
| | 5,8,11,14,17-pentazacycloicosane-4-carbonyl]-N-[1-[(2-amino-2- |
| | oxoethyl)amino]-5-(diaminomethylideneamino)-1-oxopentan-2- |
| | yl]pyrrolidine-2-carboxamide |
| Calcitonin | CGNLSTCMLGTYTQDFNKFHTFPQTAIGVGAP (SEQ ID NO: 80) |
| DD-04107 | palmitoyl-EEMQRR (SEQ ID NO: 81) |
| Efpeglenatide | (2S)-1-[3-[2-[3-[[(5S)-5-amino-5- |
| | carboxypentyl]amino]propoxy]ethoxy]propyl]pyrrolidine-2-carboxylic |
| | acid |
| Exenatide | Exendin 3 (Heloderma horridum), 2-glycine-3-L-glutamic acid |
| Exendin-(9-39) | DLSKQMEEEAVRLFIEWLKNGGPSSGAPPPS (SEQ ID NO: 82) |
| HIP-2B | IGLHDPTQGTEPNG (SEQ ID NO: 83) |
| HS-20004 | EGTFTSDVSSYLEEEAAKEFIAWLVRGGPSSGAPPPS (SEQ ID NO: 84) |
| insulin aspart | FVNQHLC₂GSHLVEALYLVC₃GERGFFYTDKT, |
| | GIVEQC₁C₂TSIC₁SLYQLENYC₃N (SEQ ID NO: 85) |
| | Two chains linked by disulfid bonds between two C₁, C₂ or C₃ |
| insulin degludec | FVNQHLC₂GSHLVEALYLVC₃GERGFFYTPK, |
| | GIVEQC₁C₂TSIC₁SLYQLENYC₃N (SEQ ID NO: 86) |
| | Two chains linked by disulfid bonds between two C₁, C₂ or C₃ |
| insulin detemir | FVNQHLC₂GSHLVEALYLVC₃GERGFFYTPK (myristoyl), |
| | GIVEQC₁C₂TSIC₁SLYQLENYC₃N (SEQ ID NO: 87) |
| | Two chains linked by disulfid bonds between two C₁, C₂ or C₃ |
| insulin glargine | FVNQHLC₂GSHLVEALYLVC₃GERGFFYTPKTRR, |
| | GIVEQC₁C₂TSIC₁SLYQLENYC₃G (SEQ ID NO: 88) |
| | Two chains linked by disulfid bonds between two C₁, C₂ or C₃ |
| insulin glulisine | FVKQHLC₂GSHLVEALYLVC₃GERGFFYTPET, |
| | GIVEQC₁C₂TSIC₁SLYQLENYC₃N (SEQ ID NO: 89) |
| | Two chains linked by disulfid bonds between two C₁, C₂ or C₃ |
| insulin lispro | FVNQHLC₂GSHLVEALYLVC₃GERGFFYTKPT, |
| | GIVEQC₁C₂TSIC₁SLYQLENYC₃N (SEQ ID NO: 90) |
| | Two chains linked by disulfid bonds between two C₁, C₂ or C₃ |
| insulin neutral | FVNQHLC₂GSHLVEALYLVC₃GERGFFYTPKA, |
| | GIVEQC₁C₂TSIC₁SLYQLENYC₃N (SEQ ID NO: 91) |
| | Two chains linked by disulfid bonds between two C₁, C₂ or C₃ |
| insulin zinc | Insulin protamine zinc |
| liraglutide | N26-(hexadecanoyl-gamma-glutamyle)-[34-arginine]GLP-1-(7-37)- |
| (recombinant) | peptide |
| Lixisenatide | HGEGTFTSDLSKQMEEEAVRLFIEWLKNGGPSSGAPPSKKKKKK (SEQ ID NO: 92) |
| NN-9535 | Glycine, L-histidyl-2-methylalanyl-L-alpha-glutamylglycyl-L-threonyl-L- |
| | phenylalanyl-L-threonyl-L-seryl-L-alpha-aspartyl-L-valyl-L-seryl-L- |
| | seryl-L-tyrosyl-L-leucyl-L-alpha-glutamylglycyl-L-glutaminyl-L-alanyl-L- |
| | alanyl-N6-[N-(17-carboxy-1-oxoheptadecyl-L-gamma-glutamyl[2-(2- |
| | aminoethoxy)ethoxy]acetyl[2-(2-aminoethoxy)ethoxy]acetyl]-L-lysyl-L- |
| | alpha-glutamyl-L-phenylalanyl-L-isoleucyl-L-alanyl-L-tryptophyl-L- |
| | leucyl-L-valyl-L-arginylglycyl-L-arginyl- |
| NN-9536 | Glycine, L-histidyl-2-methylalanyl-L-alpha-glutamylglycyl-L-threonyl-L- |
| | phenylalanyl-L-threonyl-L-seryl-L-alpha-aspartyl-L-valyl-L-seryl-L- |
| | seryl-L-tyrosyl-L-leucyl-L-alpha-glutamylglycyl-L-glutaminyl-L-alanyl-L- |
| | alanyl-N6-[N-(17-carboxy-1-oxoheptadecyl-L-gamma-glutamyl[2-(2- |
| | aminoethoxy)ethoxy]acetyl[2-(2-aminoethoxy)ethoxy]acetyl]-L-lysyl-L- |
| | alpha-glutamyl-L-phenylalanyl-L-isoleucyl-L-alanyl-L-tryptophyl-L- |
| | leucyl-L-valyl-L-arginylglycyl-L-arginyl- |
| OG-217SC | Glycine, L-histidyl-2-methylalanyl-L-alpha-glutamylglycyl-L-threonyl-L- |
| | phenylalanyl-L-threonyl-L-seryl-L-alpha-aspartyl-L-valyl-L-seryl-L- |
| | seryl-L-tyrosyl-L-leucyl-L-alpha-glutamylglycyl-L-glutaminyl-L-alanyl-L- |
| | alanyl-N6-[N-(17-carboxy-1-oxoheptadecyl-L-gamma-glutamyl[2-(2- |
| | aminoethoxy)ethoxy]acetyl[2-(2-aminoethoxy)ethoxy]acetyl]-L-lysyl-L- |
| | alpha-glutamyl-L-phenylalanyl-L-isoleucyl-L-alanyl-L-tryptophyl-L- |
| | leucyl-L-valyl-L-arginylglycyl-L-arginyl- |
| Pasireotide | [(3S,6S,9S,12R,15S,18S,20R)-9-(4-aminobutyl)-3-benzyl-12-(1H-indol- |
| | 3-ylmethyl)-2,5,8,11,14,17-hexaoxo-15-phenyl-6-[(4- |
| | phenylmethoxyphenyl)methyl]-1,4,7,10,13,16- |
| | hexazabicyclo[16.3.0]henicosan-20-yl]N-(2-aminoethyl)carbamate |
| PEX-168 | HGEGTFTSDLSKQMEEEAVRLFIEWLKNGGPSSGAPPPS (PEG) (SEQ ID NO: 93) |
| Pexiganan | GIGKFLKKAKKFGKAFVKLLKK (SEQ ID NO: 94) |
| PI-406 | Insulin (human), 28B-L-aspartic acid- |
| PMZ-2123 | DPVLW (SEQ ID NO: 95) |
| pramlintide | Amylin (human), 25-L-proline-28-L-proline-29-L-proline- |
| protamine zinc | Insulin protamine zinc |
| insulin | |
| PT-302 | l-histidylglycyl-l-glutamylglycyl-l-threonyl-l-phenylalanyl-l-threonyl-l- |
| | seryl-l-aspartyl-l-leucyl-l-seryl-l-lysyl-l-glutaminyl-l-methionyl-l- |
| | glutamyl-l-glutamyl-lglutamyl-l-alanyl-l-valyl-l-arginyl-l-leucyl-l- |
| | phenylalanyl-l-isoleucyl-l-glutamyl-l-tryptophyl-l-leucyl-l-lysyl-l- |
| | asparaginylglycylglycyl-l-prolyl-l-seryl-l-serylglycyl-l-alanyl-l-prolyl- |
| | l-prolyl-l-prolyl-l-serinamide |
| Saxenda | N26-(hexadecanoyl-gamma-glutamyle)-[34-arginine]GLP-1-(7-37)- |
| | peptide |
| Teriparatide | SVSEIQLMHNLGKHLNSMERVEWLRKKLQDVHNF (SEQ ID NO: 96) |
| teriparatide | SVSEIQLMHNLGKHLNSMERVEWLRKKLQDVHNF, acetate (SEQ ID NO: 97) |
| acetate | |
| tesamorelin | N-[(3E)-1-oxo-3-hexenyl]- |
| acetate | YADAIFTNSYRKLGQLSARKLLQDIMSRQQGESNQERGARARL, |
| | acetate (SEQ ID NO: 98) |
| Toujeo | 21(sup A)-Glycine-30(sup B)a-L-arginine-30(sup B)b-L-arginine insulin |
| | (human) |
| VTC-G15 | LVKGR (SEQ ID NO: 99) |
| ZP-4207 | HSQGTFTSDYSKYLDRARADDFVAWLKST (SEQ ID NO: 100) |

In various musculoskeletal disorders:

| | |
|---|---|
| Vosoritide | L-prolylglycyl-(human C-type natriuretic peptide-(17- |
| | 53)-peptide (CNP-37)), cyclic-(23-39)-disulfide |

In various nutritional disorders:

| | |
|---|---|
| parathyroid | (2S)-2-amino-3-[3-(hydroxymethyl)-4- |
| hormone | phosphonooxyphenyl]propanoic acid |

In various oncolodical disorders:

| | |
|---|---|
| abarelix | (2R)-2-[[(2S)-2-[[(2S)-2-[[(2R)-2-[[(2R)-2-[[(2R)-2-acetamido-3- |
| | naphthalen-2-ylpropanoyl]amino]-3-(4-chlorophenyl)propanoyl]amino]- |
| | 3-pyridin-3-ylpropanoyl]amino]-3-hydroxypropanoyl]-methylamino]-3- |
| | (4-hydroxyphenyl)propanoyl]amino]-N-[(2S)-1-[[(2S)-1-[(2S)-2-[[(2R)-1- |
| | amino-1-oxopropan-2-yl]carbamoyl]pyrrolidin-1-yl]-1-oxo-6-(propan-2- |
| | ylamino)hexan-2-yl]amino]-4-methyl-1-oxopentan-2-yl]butanediamide |
| AD-01 | QIRQQPRDPPTETLELEVSPDPAS (SEQ ID NO: 101) |
| Adipotide | CKGGRAKDCGGKLAKLAKKLAKLAK (SEQ ID NO: 102) |
| Antineoplaston | Antineoplaston A10: Benzeneacetamide, N-(2,6-dioxo-3-piperidinyl)-, |
| Therapy | (S)-Antineoplaston AS2-1: L-Glutamine, N2-(phenylacetyl)-, |
| | monosodium salt, mixt. with sodium benzeneacetate |
| ATSP-7041 | LTFEYWAQXSAA (X: Cba) (SEQ ID NO: 103) |
| BMTP-11 | CGRRAGGSCGGDKLAKLAKKLAKLAK (SEQ ID NO: 104) |
| Box-5 | MDGCEL (SEQ ID NO: 105) |
| Buserelin | acetic acid; (2S)-N-[(2S)-1-[[(2S)-1-[[(2S)-1-[[(2S)-1-[[(2R)-1-[[(2S)-1- |
| Acetate | [[(2S)-5-(diaminomethylideneamino)-1-[(2S)-2- |
| | (ethylcarbamoyl)pyrrolidin-1-yl]-1-oxopentan-2-yl]amino]-4-methyl-1- |
| | oxopentan-2-yl]amino]-3-[(2-methylpropan-2-yl)oxy]-1-oxopropan-2- |
| | yl]amino]-3-(4-hydroxyphenyl)-1-oxopropan-2-yl]amino]-3-hydroxy-1- |
| | oxopropan-2-yl]amino]-3-(1H-indol-3-yl)-1-oxopropan-2-yl]amino]-3- |
| | (1H-imidazol-5-yl)-1-oxopropan-2-yl]-5-oxopyrrolidine-2-carboxamide |
| CBP-501 | D-Arginine, 4-benzoyl-D-phenylalanyl-D-seryl-D-tryptophyl-D-seryl- |
| | 2,3,4,5,6-pentafluoro-D-phenylalanyl-3-cyclohexyl-D-alanyl-D-arginyl- |
| | D-arginyl-D-arginyl-D-glutaminyl-D-arginyl |
| CMS-024 | LYS |
| Degarelix | D-Alaninamide, N-acetyl-3-(naphtalen-2-yl)-D-alanyl-4-chloro-D- |
| Acetate | phenylalanyl-3-(pyridin-3-yl)-D-alanyl-L-seryl-4-((((4S)-2,6- |
| | dioxohexahydropyrimidin-4-yl)carbonyl)amino)-L-phenylalanyl-4- |
| | (carbamoylamino)-D-phenylalanyl-L-leucyl-N6-(1-methylethyl)-L-lysyl- |
| | L-prolyl-, acetate |
| Dolcanatide | NDECELCVNVACTGCL (SEQ ID NO: 106) |
| Dusquetide | RIVPA (SEQ ID NO: 107) |
| Edotreotide | 2-[4-[2-[[(2R)-1-[[(4R,7S,10S,13R,16S,19R)-10-(4-aminobutyl)-4- |
| | [[(2R,3R)-1,3-dihydroxybutan-2-yl]carbamoyl]-7-[(1R)-1-hydroxyethyl]- |
| | 16-[(4-hydroxyphenyl)methyl]-13-(1H-indol-3-ylmethyl)-6,9,12,15,18- |
| | pentaoxo-1,2-dithia-5,8,11,14,17-pentazacycloicos-19-yl]amino]-1-oxo- |
| | 3-phenylpropan-2-yl]amino]-2-oxoethyl]-7,10-bis(carboxymethyl)- |
| | 1,4,7,10-tetrazacyclododec-1-yl]acetic acid |
| EPI-506 | 3-[4-[2-[4-(3-chloro-2-hydroxypropoxy)phenyl]propan-2- |
| | yl]phenoxy]propane-1,2-diol |
| Foxy-5 | MDGCEL (SEQ ID NO: 108) |
| GO-2032c | RRRRRRRRRCQCRRKN (SEQ ID NO: 109) |
| Goserelin | (2S)-N-[(2S)-1-[[(2S)-1-[[(2S)-1-[[(2S)-1-[[(2R)-1-[[(2S)-1-[[(2S)-1-[(2S)- |
| | 2-[(carbamoylamino)carbamoyl]pyrrolidin-1-yl]-5- |
| | (diaminomethylideneamino)-1-oxopentan-2-yl]amino]-4-methyl-1- |
| | oxopentan-2-yl]amino]-3-[(2-methylpropan-2-yl)oxy]-1-oxopropan-2- |
| | yl]amino]-3-(4-hydroxyphenyl)-1-oxopropan-2-yl]amino]-3-hydroxy-1- |
| | oxopropan-2-yl]amino]-3-(1H-indol-3-yl)-1-oxopropan-2-yl]amino]-3- |
| | (1H-imidazol-5-yl)-1-oxopropan-2-yl]-5-oxopyrrolidine-2-carboxamide |
| LTX-315 | KKWWKKWXK; X: Dip (SEQ ID NO: 110) |
| LY-2510924 | FYKRXGEK; X: NaI (SEQ ID NO: 111) |
| Ozarelix | (2S)-1-[(2S)-2-[[(2S)-2-[[(2R)-2-[[(2S)-2-[[(2S)-2-[[(2R)-2-[[(2R)-2- |
| | [[(2R)-2-acetamido-3-naphthalen-2-ylpropanoyl]amino]-3-(4- |
| | chlorophenyl)propanoyl]amino]-3-pyridin-3-ylpropanoyl]amino]-3- |
| | hydroxypropanoyl]-methylamino]-3-(4- |
| | hydroxyphenyl)propanoyl]amino]-6- |
| | (carbamoylamino)hexanoyl]amino]hexanoyl]amino]-5- |
| | (diaminomethylideneamino)pentanoyl]-N-[(2R)-1-amino-1-oxopropan- |
| | 2-yl]pyrrolidine-2-carboxamide |
| Plitidepsin | (2S)-N-[(2R)-1-[[(3S,6R,8S,12S,16R,17S,23S)-13-[(2S)-butan-2-yl]- |
| | 12-hydroxy-20-[(4-methoxyphenyl)methyl]-6,17,21-trimethyl-3-(2- |
| | methylpropyl)-2,5,7,10,15,19,22-heptaoxo-8-propan-2-yl-9,18-dioxa- |
| | 1,4,14,21-tetrazabicyclo[21.3.0]hexacosan-16-yl]amino]-4-methyl-1- |
| | oxopentan-2-yl]-N-methyl-1-(2-oxopropanoyl)pyrrolidine-2- |
| | carboxamide |
| Romidepsin | (1S,7Z,10S,16E,21R)-7-ethylidene-4,21-di(propan-2-yl)-2-oxa-12,13- |
| | dithia-5,8,20,23-tetrazabicyclo[8.7.6]tricos-16-ene-3,6,9,19,22-pentone |
| SORC-13 | KIEFLHPSKVDLPR (SEQ ID NO: 112) |
| SPI-1620 | L-Tryptophan, N-(3-carboxy-1-oxopropyl)-L-alanyl-L-alpha-glutamyl-L- |
| | alpha-glutamyl-L-alanyl-L-valyl-L-tyrosyl-L-tyrosyl-L-alanyl-L-histidyl-L- |
| | leucyl-L-alpha-aspartyl-L-isoleucyl-L-isoleucyl- |

In various gastrointestinal disorders:

| | |
|---|---|
| B27-PD | ALNEDLSSWTAADT (SEQ ID NO: 113) |
| C-16Y | DFKLFAVYIKYR (SEQ ID NO: 114) |
| RGN-259 | MSDKPDMAEIEKFDKSKLKKTETQEKNPLPSKETIEQEKQ |
| | AGES (SEQ ID NO: 115) |
| Spaglumic | (2S)-2-[[(2S)-2-acetamido-3- |
| Acid | carboxypropanoyl]amino]pentanedioic acid |

In various functional gastrointestinal disorders:

| | |
|---|---|
| Nepadutant | (2S)-2-[[(3S,6S,9S,12S)-12-[[(2S)-4-[[(2R,3R,4R,5S,6R)- |
| | 3-acetamido-4, 5-dihydroxy-6-(hydroxymethyl)oxan-2- |
| | yl]amino]-2-amino-4-oxobutanoyl]amino]-6-benzyl-9-(1H- |
| | indol-3-ylmethyl)-5,8,11,14-tetraoxo-1,4,7,10- |
| | tetrazacyclotetradecane-3-carbonyl]amino]-4- |
| | methylpentanoic acid |

In various respiratory disorders:

| | |
|---|---|
| CGEN-25009 | GQKGQVGPPGAAVRRAYAAFSVGRRAYAAFSV |
| | (SEQ ID NO: 116) |
| Lucinactant | KLLLLKLLLLKLLLLKLLLLK-surfactant |
| | (SEQ ID NO: 117) |
| MMI-0100 | YARAAARQARAKALARQLGVAA |
| | (SEQ ID NO: 118) |

In various women's health disorders:

| | |
|---|---|
| Atosiban | (2S)-N-[(2S)-5-amino-1-[(2-amino-2-oxoethyl)amino]-1-oxopentan-2- |
| | yl]-1-[(4R,7S,13S,16R)-7-(2-amino-2-oxoethyl)-13-[(2S)-butan-2-yl]-16- |
| | [(4-ethoxyphenyl)methyl]-10-[(1R)-1-hydroxyethyl]-6,9,12,15,18- |
| | pentaoxo-1,2-dithia-5,8,11,14,17-pentazacycloicosane-4- |
| | carbonyl]pyrrolidine-2-carboxamide |
| Barusiban | (4S,7S,10S,16R)-N-[(2S)-5-amino-1-hydroxypentan-2-yl]-7-(2-amino-2- |
| | oxoethyl)-13-[(2S)-butan-2-yl]-10-[(2R)-butan-2-yl]-16-(1H-indol-3- |
| | ylmethyl)-N-methyl-6,9,12,15,18-pentaoxo-1-thia-5,8,11,14,17- |
| | pentazacycloicosane-4-carboxamide |
| Bremelanotide | L-Lysine, N-acetyl-L-norleucyl-L-a-aspartyl-L-histidyl-D-phenylalanyl-L- |
| | arginyl-L-tryptophyl-, (2-7)-lactam |
| Carbetocin | N-[1-[(2-amino-2-oxoethyl)amino]-4-methyl-1-oxopentan-2-yl]-1-[6-(2- |
| | amino-2-oxoethyl)-9-(3-amino-3-oxopropyl)-12-butan-2-yl-15-[(4- |
| | methoxyphenyl)methyl]-5,8,11,14,17-pentaoxo-1-thia-4,7,10,13,16- |
| | pentazacycloicosane-3-carbonyl]pyrrolidine-2-carboxamide |
| Cetrorelix | (2S)-1-[(2S)-2-[[(2S)-2-[[(2R)-2-[[(2S)-2-[[(2S)-2-[[(2R)-2-[[(2R)-2- |
| | [[(2R)-2-acetamido-3-naphthalen-2-ylpropanoyl]amino]-3-(4- |
| | chlorophenyl)propanoyl]amino]-3-pyridin-3-ylpropanoyl]amino]-3- |
| | hydroxypropanoyl]amino]-3-(4-hydroxyphenyl)propanoyl]amino]-5- |
| | (carbamoylamino)pentanoyl]amino]-4-methylpentanoyl]amino]-5- |
| | (diaminomethylideneamino)pentanoyl]-N-[(2R)-1-amino-1-oxopropan- |
| | 2-yl]pyrrolidine-2-carboxamide |
| Cetrorelix | (2S)-1-[(2S)-2-[[(2S)-2-[[(2R)-2-[[(2S)-2-[[(2S)-2-[[(2R)-2-[[(2R)-2- |
| Acetate | [[(2R)-2-acetamido-3-naphthalen-2-ylpropanoyl]amino]-3-(4- |
| | chlorophenyl)propanoyl]amino]-3-pyridin-3-ylpropanoyl]amino]-3- |
| | hydroxypropanoyl]amino]-3-(4-hydroxyphenyl)propanoyl]amino]-5- |
| | (carbamoylamino)pentanoyl]amino]-4-methylpentanoyl]amino]-5- |
| | (diaminomethylideneamino)pentanoyl]-N-[(2R)-1-amino-1-oxopropan- |
| | 2-yl]pyrrolidine-2-carboxamide; acetic acid |
| Follitropin | 1-[19-amino-7-(2-amino-2-oxoethyl)-13-butan-2-yl-10-(1-hydroxyethyl)- |
| | 16-[(4-hydroxyphenyl)methyl]-6,9,12,15,18-pentaoxo-1,2-dithia- |
| | 5,8,11,14,17-pentazacycloicosane-4-carbonyl]-N-[1-[(2-amino-2- |
| | oxoethyl)amino]-4-methyl-1-oxopentan-2-yl]pyrrolidine-2-carboxamide |
| Ganirelix | (2S)-1-[(2S)-2-[[(2S)-2-[[(2R)-2-[[(2S)-2-[[(2S)-2-[[(2R)-2-[[(2R)-2- |
| acetate | [[(2R)-2-acetamido-3-naphthalen-2-ylpropanoyl]amino]-3-(4- |
| | chlorophenyl)propanoyl]amino]-3-pyridin-3-ylpropanoyl]amino]-3- |
| | hydroxypropanoyl]amino]-3-(4-hydroxyphenyl)propanoyl]amino]-6- |
| | [bis(ethylamino)methylideneamino]hexanoyl]amino]-4- |
| | methylpentanoyl]amino]-6-[bis |
| | (ethylamino)methylideneamino]hexanoyl]-N-[(2R)-1-amino-1- |
| | oxopropan-2-yl]pyrrolidine-2-carboxamide; acetic acid |
| Gonadorelin | XHWSYGLRPG X: Pyroglutamic acid (SEQ ID NO: 119) |
| Merotocin | Glycinamide, N-(4-mercapto-1-oxobutyl)-L-tyrosyl-L-isoleucyl-L- |
| | glutaminyl-L-asparaginyl-L-cysteinyl-N-((4-fluorophenyl)methyl)glycyl- |
| | L-leucyl-, cyclic (1->5)-thioether |
| Nafarelin | X₁HWSYXLRPG (SEQ ID NO: 120) X₁: Pyroglutamic acid, X: NaI |
| Oxytocin | CYIQNCPLG (SEQ ID NO: 121) |
| Urofollitropin | CYITNCPLG (SEQ ID NO: 122) |

In another aspect, a peptide analog of the present invention may be used in an in vitro or in vivo diagnostic method. In the present invention, the in vitro or in vivo diagnostic method may be any method known to one skilled in the art in which a peptide analog could be used. For example said diagnostic methods may be selected from the group comprising medical imaging methods such as Single Photon Emission Computed Tomography (SPECT), Positron Emission Tomography (PET), Contrast enhanced ultrasound imaging, and Magnetic Resonance Imaging (MRI) by using for example Mangradex nanoparticles.

The present invention will be further illustrated by the following examples. However these examples should not be interpreted in any way as limiting the scope of the present invention.

### EXAMPLES

### Example 1: General Material and Methods

Reagents were obtained from commercial source and used without any further purification. Fmoc-L-amino acids were purchased from Novabiochem, Polypeptides and Iris Biotech. Fmoc-protected Rink Amide NovaGel® resin was purchased from Novabiochem and the overall yields for the solid-phase syntheses were calculated based on the initial loadings provided by the supplier (0.7 mmol/g). Fmoc-protected Wang NovaGel® resin was purchased from Novabiochem and the overall yields for the solid-phase syntheses were calculated based on the initial loadings provided by the supplier (0.1 mmol/g).

### Analytical Reverse-Phase High Performance Liquid Chromatography (RP-HPLC) Analysis

Analysis were performed either on a C18 Sunfire column (5 μm, 4.6 mm×150 mm) using a linear gradient (5% to 95% in 20 min, flow rate of 1 mL·min⁻¹) of solvent B (0.1% TFA in CH₃CN, v/v) in solvent A (0.1% TFA in H₂O, v/v). Detection was set AT 220 nm and 254 nM.

### Semi-Preparative RP-HPLC Chromatography Purifications

Purifications were performed on Sunfire C18 column (5 μm, 19×150 mm) on Gilson PLC2020 with absorption detection. The separation was achieved using successive isocratic and linear gradients (5 min at 5%; 5% to 60% in 30 min; 60% to 100% in 10 min; flow rate of 20 mL·min⁻¹) of solvent B (0.1% TFA in CH₃CN, v/v) in solvent A (0.1% TFA in H₂O, v/v).

### Liquid Chromatography Mass Spectra (LC-MS) Analysis

Analysis were obtained on a ZQ (Z quadripole) Waters/Micromass spectrometer equipped with an X-Terra C18 column (0.5 μm, 4.6 mm×50 mm) using electrospray ionization mode (ESI).

### High Resolution Mass Spectra (HR-MS) Analysis

Analysis were acquired on a Bruker MicroTof mass spectrometer, using electrospray ionization (ESI) and a time-of-flight analyzer (TOF) or on an Autoflex II TOF/TOF Bruker mass spectrometer using matrix-assisted laser desorption/ionization technique (MALDI) and a time-of-light analyzer (TOF).

### General Protocol for Standard Automated Solid-Phase Peptide Synthesis (SPPS)

Standard automated solid-phase peptide synthesis (SPPS) were performed on an Applied Biosystem ABI 433A synthesizer (Appelar, France). The elongation was carried out by coupling a 10-fold excess of Fmoc-L-amino acid derivatives, using 2-(1H-benzotriazol-1-yl)-1,1,3,3-tetramethyluronium hexafluorophosphate (HBTU), 1-hydroxybenzotriazole (HOBt), and diisopropylethylamine (Hünig's base) (DIPEA) as coupling reagents in N,N-dimethylformamide (DMF) as solvent. At the end of the peptide sequence synthesis, the resin was washed with CH₂Cl₂ and MeOH and then dried in vacuo. After each coupling step, Fmoc deprotection was performed by treatment with piperidine monitored by UV at 301 nm.

### General Protocol for Peptide Elongation by Manual SPPS

Peptide elongation was performed starting from fried resin previously synthetized by standard automated SPPS. Non-automated SPPS were performed in polypropylene tubes equipped with polyethylene frits and polypropylene caps using an orbital agitator shaking device.

The Fmoc-protected resin (1 equiv) was swollen 1 h in DMF and the excess solvent was removed by filtration. Cleavage of the Fmoc protecting group was performed in a solution of 20% (v/v) piperidine in DMF (2 times for 15 min). The piperidine solution was drained off and the resin was washed with successively DMF, CH₂Cl₂ and MeOH (3×0.5 mL).

All Fmoc-protected amino acids (4 equiv) were coupled in N,N-dimethylformamide (DMF) for 45 min using HBTU (3.8 equiv) and HOBt (4 equiv) with N,N-diisopropylethylamine (DIEA) (12 equiv) as activating agents. The excess solvent was removed by filtration and the resin was washed with successively DMF, CH₂Cl₂ and MeOH (3×0.5 mL).

The cycle of coupling, washing and deprotection were repeated until the targeted peptides were obtained. The completion of couplings and Fmoc deprotections were monitored with ninhydrin test and TNBS test:

Ninhydrin test (for primary amines): Resin beads were suspended in 2 drops of a solution containing 5 g of ninhydrin dissolved in 100 mL of ethanol, 2 drops of a solution containing 80 g of liquefied phenol in 20 mL of ethanol, and 2 drops of a 0.001M aqueous solution of potassium cyanide to 98 mL pyridine. The mixture was heated at 100° C. for 1 min. The color positive test (presence of free amino groups). A yellow or blue solution and yellow beads indicate a negative test.

TNBS test (for primary amines): Resin beads were suspended in 2 drops of a solution containing 10% (v/v) DIPEA in DMF and 2 drops of a solution containing 2,4,6-trinitrobenzenesulfonic acid (TNBS) in DMS. The color of the solution and the beads were observed. A yellow-red solution and red beads indicate a positive test. A yellow-red solution and yellow beads indicate a negative test.

General Protocol for Peptide Elongation with a Perfluoroalkyl Chain

The resin containing the peptide sequence of interest (1 equiv) was swollen in DMF, and the excess solvent was removed by filtration. A solution of piperidine in DMF (20% v/v—0.5 mL) was added, and the mixture was shaken at room temperature for 15 min. The solution was drained, and the operation was repeated for 15 min. The solution was drained, and the resin was washed with DMF and CH₂Cl₂. In a separate vial, DIEA (8 equiv) was added to a solution of 4,4,5,5,6,6,7,7,8,8,9,9,10,10,11,11-heptadecafluoroundecanoic acid (2 equiv), HBTU (2 equiv), and HOBt (1.9 equiv) in DMF (0.5 mL). The mixture was stirred at room temperature for 1 min and was added to the resin. The mixture was shaken at room temperature for 2 h. The solution was drained and the resin was washed with DMF, CH₂Cl₂, and MeOH the dried in vacuo.

### General Protocol for Resin Cleavage

The dried resin was treated with TFA/Phenol/Thioanisole/1,2-Ethanedithiol/water (10 mL/0.75 g/0.5 mL/0.25 mL/0.5 mL) and the mixture was shaken at room temperature for 3 h. The filtrate was collected in a cold diethyl ether solution and the beads washed with TFA. The solution was centrifuged at 3000 rpm for 2 min. The precipitate was washed in a cold diethyl ether solution and centrifuged at 3000 rpm for 2 min. The diethyl ether solution was eliminated and the precipitate was dried in vacuo. The crude product was purified by semi-preparative RP-HPLC and lyophilized.

### Example 2: Peptide Analogs Synthesized and their Characterization

### Apelin Analogs

### Synthesis of LE-122 and LE-124

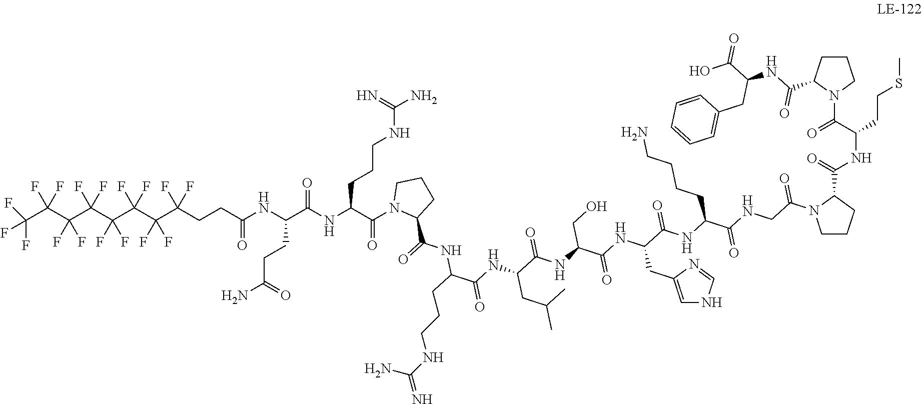 [SMILES: CSCC[C@H](NC(=O)[C@@H]1CCCN1C(=O)CNC(=O)[C@H](CCCCN)NC(=O)[C@H](Cc1c[nH]cn1)NC(=O)[C@H](CO)NC(=O)[C@H](CC(C)C)NC(=O)C(CCCNC(=N)N)NC(=O)[C@@H]1CCCN1C(=O)[C@H](CCCNC(=N)N)NC(=O)[C@H](CCC(N)=O)NC(=O)CCC(F)(F)C(F)(F)C(F)(F)C(F)(F)C(F)(F)C(F)(F)C(F)(F)C(F)(F)F)C(=O)N1CCC[C@H]1C(=O)N[C@@H](Cc1ccccc1)C(=O)O]

Fmoc-Gln-Arg-Pro-Arg-Leu-Ser-His-Lys-Gly-Pro-Met-Pro-Phe-Wang resin (16 μmol), 4,4,5,5,6,6,7,7,8,8,9,9,10,10,11,11-heptadecafluoroundecanoic acid (2 equiv), HBTU (2 equiv), HOBt (1.9 equiv) and DIEA (8 equiv) were reacted according the general protocol, affording the title compound (18.9 mg, 47.7%) as a white solid. t_{R}=11.05 min (>95% purity at 220 nm); HRMS (ESI) calcd for C₈₀H₁₁₄F₁₇N₂₃O₁₇₅: 2023.82123; found: 2023.82752.

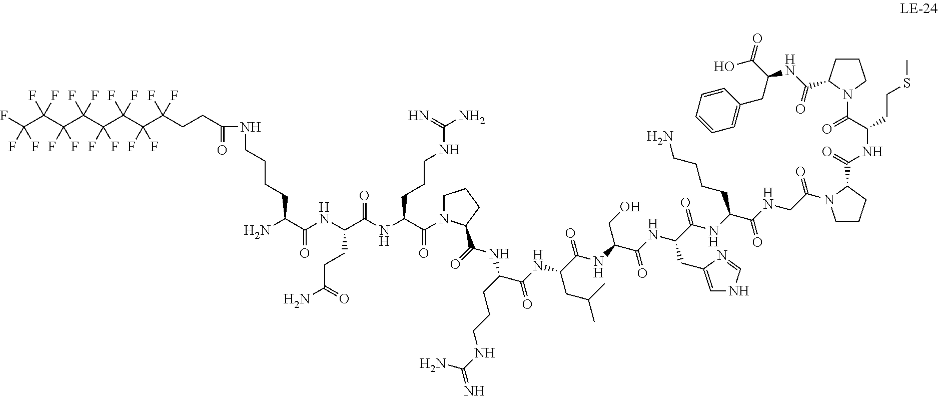 [SMILES: CSCC[C@H](NC(=O)[C@@H]1CCCN1C(=O)CNC(=O)[C@H](CCCCN)NC(=O)[C@H](Cc1c[nH]cn1)NC(=O)[C@H](CO)NC(=O)[C@H](CC(C)C)NC(=O)[C@H](CCCNC(=N)N)NC(=O)[C@@H]1CCCN1C(=O)[C@H](CCCNC(=N)N)NC(=O)[C@H](CCC(N)=O)NC(=O)[C@@H](N)CCCCNC(=O)CCC(F)(F)C(F)(F)C(F)(F)C(F)(F)C(F)(F)C(F)(F)C(F)(F)C(F)(F)F)C(=O)N1CCC[C@H]1C(=O)N[C@@H](Cc1ccccc1)C(=O)O]

Fmoc-Gln-Arg-Pro-Arg-Leu-Ser-His-Lys-Gly-Pro-Met-Pro-Phe-Wang resin (16 μmol), Boc-Lys(Fmoc)-OH (4 equiv), HBTU (3.8 equiv), HOBt (4 equiv) and DIEA (12 equiv) were reacted according the general procedure. Then, 4,4,5,5,6,6,7,7,8,8,9,9,10,10,11,11-heptadecafluoroundecanoic acid (2 equiv), HBTU (2 equiv), HOBt (1.9 equiv) and DIEA (8 equiv) were reacted according the general protocol, affording the title compound (14.5 mg, 35%) as a white solid. t_{R}=10.68 min (>95% purity at 220 nm); HRMS (ESI) calcd for C₈₆H₁₂₆F₁₇N₂₅O₁₈S: 2151.91619; found: 2151.91393.

### Characterization

### Affinity

Evaluation of the agonist activity of compounds at the human APJ receptor expressed in transfected CHO cells, determined by measuring their effects on cAMP modulation using the HTRF detection method. Experimental protocol: The cells are suspended in HBSS buffer (Invitrogen) complemented with 20 mM HEPES (pH 7.4) and 500 μM IBMX, then distributed in microplates at a density of 1.5×104 cells/well in the presence of either of the following: HBSS (basal control), the reference agonist at 30 nM (stimulated control) or various concentrations (EC50 determination), or the test compounds. Thereafter, the adenylyl cyclase activator NKH 477 is added at a final concentration of 0.3 μM. Following 10 min incubation at 37° C., the cells are lysed and the fluorescence acceptor (D2-labeled cAMP) and fluorescence donor (anti-cAMP antibody labeled with europium cryptate) are added. After 60 min at room temperature, the fluorescence transfer is measured at λex=337 nm and λem=620 and 665 nm using a microplate reader (Envison, Perkin Elmer). The cAMP concentration is determined by dividing the signal measured at 665 nm by that measured at 620 nm (ratio). The results are expressed as a percent of the control response to 30 nM apelin-13. The standard reference agonist is apelin-13, which is tested in each experiment at several concentrations to generate a concentration-response curve from which its EC50 value is calculated.

### IC50

Evaluation of the affinity of compounds for the human apelin receptor in transfected CHO cells determined in a radioligand binding assay. Experimental protocol: Cell membrane homogenates (1 μg protein) are incubated for 120 min at 22° C. with 0.03 nM [125I](Glp65, Nle75, Tyr77)-apelin-13 in the absence or presence of the test compound in a buffer containing 50 mM Hepes/NaOH (pH 7.4), 100 mM NaCl, 10 mM KCl, 5 mM MgCl2, 1 mM EDTA, 0.04% bacitracin and 0.1% BSA. Nonspecific binding is determined in the presence of 1 μM apelin-13. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard). The results are expressed as a percent inhibition of the control radioligand specific binding. The standard reference compound is apelin-13, which is tested in each experiment at several concentrations to obtain a competition curve from which its IC50 is calculated.

### Solubility Study

The solubility of each fluoropeptide was evaluated after dissolution in water to reach 100 μM. The resulting solution was vortexed 1 min following by 1 min in bath sonication. Solubility was then assessed by visual observation of the resulting dispersion (Clear/Cloudy and presence of particulates).

### Human Plasma Stability

This procedure is designed to determine the stability of a test compound in blood or plasma from human or animal species in a 96-well plate format. The test compound is quantified at 5 time points by HPLC-MS/MS analysis.

Test concentration: 1 μM with a final DMSO concentration of 0.5%. Experimental protocol: Blood or plasma are pre-warmed at 37° C. water bath for 5 min, followed by addition of the test compound. The incubation is performed in a 37° C. water bath for 2 h. An aliquot of the incubation mixture is transferred to acetonitrile at 0, 0.5, 1, 1.5 and 2 h, respectively. Samples are then mixed and centrifuged. Supernatants are used for HPLC-MS/MS analysis. Reference compounds Propoxycaine and propantheline are tested simultaneously with the test compound in each assay. Analytical methods Samples are analyzed by HPLC-MS/MS using selected reaction monitoring. The HPLC system consists of a binary LC pump with autosampler, a C-18 column, and a gradient. Conditions may be adjusted as necessary. Data analysis Peak areas corresponding to the test compound are recorded. The compound remaining (%) is calculated by comparing the peak area at each time point to time zero. The half-life is calculated from the slope of the initial linear range of the logarithmic curve of compound remaining (%) vs. time, assuming first order kinetics

The results are presented in the table below:

human APJ (apelin) receptor

| | | | | Human |
|---|---|---|---|---|
| | Affinity | IC50 | Solubility | plasma |
| | % of inhibition | cAMP | in water | stability |
| Peptide | at 10 μM | (nM) | (μM) | (t_{1/2}, min) |
| pE13F | 101.2 | 24 | >100 | 5 |
| LE-122 | 100.0 | 2.7 | >100 | 1241 |
| LE-124 | 101.3 | 1.3 | — | 357 |

These results demonstrate that the incorporation of the fluorocarbon chain has no impact on both the binding affinity and the functional activity of the resulting peptides, regardless of the location of the fluorocarbon chain, either on the epsilon or on the alpha-amino group of the apelin peptide. Noteworthy, the solubility of both fluoropeptide is above 100 μM in water. Finally, the location of the chain has an impact on the metabolic stability of the fluoropeptide in human plasma. Indeed, the more stable construct is obtained when the fluorocarbon chain is introduced on the alpha-amino part of the peptide (LE-122, t_{1/2}>1241 min).

### Angiotensin II Analogs

### Synthesis of LE-120

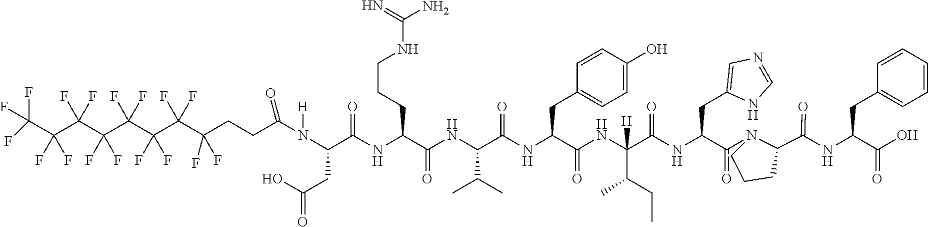 [SMILES: CC[C@H](C)[C@H](NC(=O)[C@H](Cc1ccc(O)cc1)NC(=O)[C@@H](NC(=O)[C@H](CCCNC(=N)N)NC(=O)[C@H](CC(=O)O)NC(=O)CCC(F)(F)C(F)(F)C(F)(F)C(F)(F)C(F)(F)C(F)(F)C(F)(F)C(F)(F)F)C(C)C)C(=O)N[C@@H](Cc1cnc[nH]1)C(=O)N1CCC[C@H]1C(=O)N[C@@H](Cc1ccccc1)C(=O)O]

Fmoc-Asp-Arg-Val-Tyr-Ile-His-Pro-Phe-Wang resin (50 μmol), 4,4,5,5,6,6,7,7,8,8,9,9,10,10,11,11-heptadecafluoroundecanoic acid (2 equiv), HBTU (2 equiv), HOBt (1.9 equiv) and DIEA (8 equiv) were reacted according the general protocol, affording the title compound (41.4 mg, 47%) as a white solid. t_{R}=12.94 min (>95% purity at 220 nm); HRMS (ESI) calcd for C₆₁H₁₇₄F₁₇N₁₃O₁₃: 15109.52576; found: 1519.52309.

### Characterization

### Affinity

Evaluation of the agonist activity of compounds at the human AT1 receptor expressed in transfected HEK-293 cells, determined by measuring their effect on cytosolic Ca2+ ion mobilization using a fluorimetric detection method. Experimental protocol: The cells are suspended in DMEM buffer (Invitrogen), then distributed in microplates at a density of 4.104 cells/well. The fluorescent probe (Fluo4 Direct, Invitrogen) mixed with probenicid in HBSS buffer (Invitrogen) complemented with 20 mM Hepes (Invitrogen) (pH 7.4) is then added into each well and equilibrated with the cells for 60 min at 37° C. then 15 min at 22° C. Thereafter, the assay plates are positioned in a microplate reader (CellLux, PerkinElmer) which is used for the addition of the test compound, reference agonist or HBSS buffer (basal control), and the measurements of changes in fluorescence intensity which varies proportionally to the free cytosolic Ca2+ ion concentration. For stimulated control measurements, angiotensin-II at 30 nM is added in separate assay wells. The results are expressed as a percent of the control response to 30 nM angiotensin-II. The standard reference agonist is angiotensin-II, which is tested in each experiment at several concentrations to generate a concentration-response curve from which its EC50 value is calculated.

### IC50

Evaluation of the affinity of compounds for the human angiotensin-II AT1 receptor in transfected HEK-293 cells determined in a radioligand binding assay. Experimental protocol: Cell membrane homogenates (8 μg protein) are incubated for 120 min at 37° C. with 0.05 nM [125I][Sar1-Ile8]angiotensin-II in the absence or presence of the test compound in a buffer containing 50 mM Tris-HCl (pH 7.4), 5 mM MgCl2, 1 mM EDTA and 0.1% BSA. Nonspecific binding is determined in the presence of 10 μM angiotensin II. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard). The results are expressed as a percent inhibition of the control radioligand specific binding. The standard reference compound is saralasin, which is tested in each experiment at several concentrations to obtain a competition curve from which its IC50 is calculated.

### Solubility Study

The solubility of the fluoropeptide was evaluated after dissolution in water to reach 100 μM. The resulting solution was vortexed 1 min following by 1 min in bath sonication. Solubility was then assessed by visual observation of the resulting dispersion (Clear/Cloudy and presence of particulates).

### Human Plasma Stability

This procedure is designed to determine the stability of a test compound in blood or plasma from human or animal species in a 96-well plate format. The test compound is quantified at 5 time points by HPLC-MS/MS analysis.

Test concentration: 1 μM with a final DMSO concentration of 0.5%. Experimental protocol: Blood or plasma are pre-warmed at 37° C. water bath for 5 min, followed by addition of the test compound. The incubation is performed in a 37° C. water bath for 2 h. An aliquot of the incubation mixture is transferred to acetonitrile at 0, 0.5, 1, 1.5 and 2 h, respectively. Samples are then mixed and centrifuged. Supernatants are used for HPLC-MS/MS analysis. Reference compounds Propoxycaine and propantheline are tested simultaneously with the test compound in each assay. Analytical methods Samples are analyzed by HPLC-MS/MS using selected reaction monitoring. The HPLC system consists of a binary LC pump with autosampler, a C-18 column, and a gradient. Conditions may be adjusted as necessary. Data analysis Peak areas corresponding to the test compound are recorded. The compound remaining (%) is calculated by comparing the peak area at each time point to time zero. The half-life is calculated from the slope of the initial linear range of the logarithmic curve of compound remaining (%) vs. time, assuming first order kinetics

The results are presented in the table below:

human AT1R

| | | | | Human |
|---|---|---|---|---|
| | Affinity | EC50 Ca²⁺ | Solubility | plasma |
| | % of inhibition | production | in water | stability |
| Peptide | at 10 μM | (nM) | (μM) | (t_{1/2}, min) |
| Angiotensin II | 100.2 | 14 | >100 | 85 |
| LE120 | 66.4 | 460 | >100 | >1440 |

These results indicate that the incorporation of the fluorocarbon chain has an impact on both the affinity and the functional activity compared to the native peptide. Indeed, the affinity of LE120 is significantly decreased altogether with the efficacy from 14 vs 460 nM, respectively. However, as previously described for apelin-13, the human plasma stability is greatly improved from 85 min for the native peptide to >1440 min for the fluoropeptide, again demonstrating the beneficial effect of the fluorocarbon chain on the metabolic stability.

### Oxytocin Analogs

### Synthesis of SR-11-74

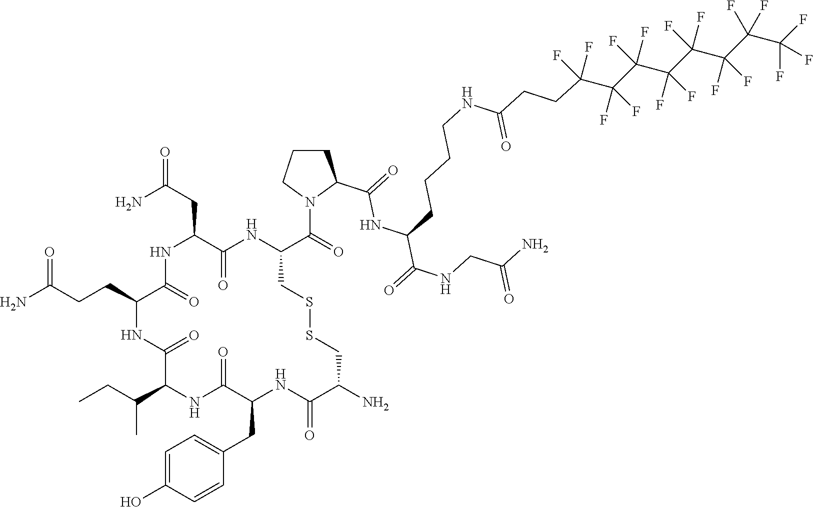 [SMILES: CCC(C)[C@@H]1NC(=O)[C@H](Cc2ccc(O)cc2)NC(=O)[C@@H](N)CSSC[C@@H](C(=O)N2CCC[C@H]2C(=O)N[C@@H](CCCCNC(=O)CCC(F)(F)C(F)(F)C(F)(F)C(F)(F)C(F)(F)C(F)(F)C(F)(F)C(F)(F)F)C(=O)NCC(N)=O)NC(=O)[C@H](CC(N)=O)NC(=O)[C@H](CCC(N)=O)NC1=O]

Fmoc-Cys-Tyr-Ile-Gln-Asn-Cys-Pro-Lys-Gly-NH₂-Rink resin (165 μmol), 4,4,5,5,6,6,7,7,8,8,9,9,10,10,11,11-heptadecafluoroundecanoic acid (2 equiv), HBTU (2 equiv), HOBt (1.9 equiv) and DIEA (8 equiv) were reacted according the general protocol, affording the title compound (21.1 mg, 8%) as a white solid. t_{R}=12.78 min (>95% purity at 220.8 nm); HRMS (ESI) calcd for C₅₄H₇₀F₁₇N₁₃O₁₃S₂: 1495.4386; found: 1495.43437.

### Characterization

### Affinity

Evaluation of the agonist activity of compounds at the human OT receptor endogenously expressed in ECV304 cells, determined by measuring their effect on cytosolic Ca2+ ion mobilization using a fluorimetric detection method. Experimental protocol: The cells are suspended in DMEM buffer (Invitrogen), then distributed in microplates at a density of 3.104 cells/well. The fluorescent probe (Fluo4 Direct, Invitrogen) mixed with probenicid in HBSS buffer (Invitrogen) complemented with 20 mM Hepes (Invitrogen) (pH 7.4) is then added into each well and equilibrated with the cells for 60 min at 37° C. then 15 min at 22° C. Thereafter, the assay plates are positioned in a microplate reader (CellLux, PerkinElmer) which is used for the addition of the test compound, reference agonist or HBSS buffer (basal control), and the measurements of changes in fluorescence intensity which varies proportionally to the free cytosolic Ca2+ ion concentration. For stimulated control measurements, oxytocin at 3 μM is added in separate assay wells. The results are expressed as a percent of the control response to 3 μM oxytocin. The standard reference agonist is oxytocin, which is tested in each experiment at several concentrations to generate a concentration-response curve from which its EC50 value is calculated.

### IC50

Evaluation of the affinity of compounds for the human vasopressin Oxytocin receptor in transfected Chem-1 cells determined in a radioligand binding assay. Experimental protocol: Cell membrane homogenates (about 10 μg protein) are incubated for 120 min at 22° C. with 0.8 nM [3H]Oxytocin in the absence or presence of the test compound in a buffer containing 50 mM Tris-HCl (pH 7.4), 5 mM MgCl2 and 0.1% BSA. Nonspecific binding is determined in the presence of 1 μM Oxytocin. Following incubation, the samples are filtered rapidly under vacuum through glass fiber filters (GF/B, Packard) presoaked with 0.3% PEI and rinsed several times with ice-cold 50 mM Tris-HCl and 0.1% BSA using a 96-sample cell harvester (Unifilter, Packard). The filters are dried then counted for radioactivity in a scintillation counter (Topcount, Packard) using a scintillation cocktail (Microscint 0, Packard). The results are expressed as a percent inhibition of the control radioligand specific binding. The standard reference compound is Oxytocin, which is tested in each experiment at several concentrations to obtain a competition curve from which its IC50 is calculated.

### Solubility Study

The solubility of the fluoropeptide was evaluated after dissolution in water to reach 100 μM. The resulting solution was vortexed 1 min following by 1 min in bath sonication. Solubility was then assessed by visual observation of the resulting dispersion (Clear/Cloudy and presence of particulates).

### Human Plasma Stability

This procedure is designed to determine the stability of a test compound in blood or plasma from human or animal species in a 96-well plate format. The test compound is quantified at 5 time points by HPLC-MS/MS analysis.

Test concentration: 1 μM with a final DMSO concentration of 0.5%. Experimental protocol: Blood or plasma are pre-warmed at 37° C. water bath for 5 min, followed by addition of the test compound. The incubation is performed in a 37° C. water bath for 2 h. An aliquot of the incubation mixture is transferred to acetonitrile at 0, 0.5, 1, 1.5 and 2 h, respectively. Samples are then mixed and centrifuged. Supernatants are used for HPLC-MS/MS analysis. Reference compounds Propoxycaine and propantheline are tested simultaneously with the test compound in each assay. Analytical methods Samples are analyzed by HPLC-MS/MS using selected reaction monitoring. The HPLC system consists of a binary LC pump with autosampler, a C-18 column, and a gradient. Conditions may be adjusted as necessary. Data analysis Peak areas corresponding to the test compound are recorded. The compound remaining (%) is calculated by comparing the peak area at each time point to time zero. The half-life is calculated from the slope of the initial linear range of the logarithmic curve of compound remaining (%) vs. time, assuming first order kinetics.

The results are presented in the table below:

human oxytocin receptor

| | | | | Human |
|---|---|---|---|---|
| | Affinity | EC50 Ca²⁺ | Solubility | plasma |
| | % of inhibition | production | in water | stability |
| Peptide | à 10 nM | (nM) | (μM) | (t_{1/2}, min) |
| Oxytocin | 102.2 | 42 | >100 | 147 |
| SR-11-74 | 91.3 | <25% at 100 | >100 | 239 |
| | | nM | | |

In the case of cyclic oxytocin, the incorporation of the fluorocarbon chain has a profound effect on the functional activity of the peptide (EC50 from 42 nM for the native peptide to >25% at 100 nM for the fluoro-ocytocin). In another hand, the presence of the fluorocarbon chain has a low impact on the human plasma stability (t_{1/2}=147 min for the oxytocin vs 239 min for the fluoro-oxytocin).

### REFERENCE LISTING

1. Fosgerau, K.; Hoffmann, T. Peptide therapeutics: current status and future directions. Drug Discovery Today 2015, 20, 122-128
2. Vlieghe, P.; Lisowski, V.; Martinez, J.; Khrestchatisky, M. Synthetic therapeutic peptides: science and market. Drug Discovery Today 2010, 15, 40-56
3. Congreve, M.; Langmead, C. J.; Mason, J. S.; Marshall, F. H. Progress in Structure Based Drug Design for G Protein-Coupled Receptors. J. Med. Chem. 2011, 54, 4283-4311
4. Narayanan S, Harris D L, Maitra R, Runyon S P. Regulation of the Apelinergic System and Its Potential in Cardiovascular Disease: Peptides and Small Molecules as Tools for Discovery. J Med Chem. 2015 Oct. 22; 58(20):7913-27
5. Marc Y, Llorens-Cortes C. The role of the brain renin-angiotensin system in hypertension: implications for new treatment. Prog Neurobiol. 2011 October; 95(2):89-103
6. Modi, M. E.; Young, L. J. The oxytocin system in drug discovery for autism: Animal models and novel therapeutic strategies. Horm. Behav. 2012, 61, 340-350

1) A metabolically stable and non-immunogenic peptide analog completely hydrosoluble at physiological pH having a peptide covalently linked to a fluorocarbon group, directly or through a linker selected from the group consisting of a PEG or a peptide having from 1 to 6 amino acids, either on the alpha-amino or the epsilon-amino group of at least one lysine of said peptide, when the linker is a lysine, the fluorocarbon group is directly linked to the epsilon-amino group of said linker;
but excluding an apelin analog having the following peptide of formula (I):
Lys-Phe-Xaa1-Arg-Xaa2-Arg-Pro-Arg-Xaa3-Ser-Xaa4-Lys-Xaa5-Pro-Xaa6-Pro-Xaa7 (I)
wherein said peptide of formula (I) is linked to a fluorocarbon group, an acetyl group, or an acyl group —C(O)R where R is a C₇₋₃₀ alkyl, directly or through a linker selected from the group consisting of PEG, lysine and arginine, either on the alpha-amino or the epsilon-amino group of at least one lysine of the peptide formula (I), and when the linker is a lysine, the fluorocarbon group, acetyl or acyl group is directly linked either on the epsilon-amino group of said linker and wherein:
Xaa1 is arginine (R) or D-isomer arginine (R_{D});
Xaa2 is glutamine (Q) or D-isomer glutamine (Q_{D})
Xaa3 is leucine (L) or D-isomer leucine (L_{D});
Xaa4 is histidine (H) or α-aminoisobutyric acid (Aib);
Xaa5 is alanine (A) or D-isomer alanine (A_{D}) or glycine (G);
Xaa6 is methionine (M) or norleucine (Nle);
Xaa7 is phenylalanine (F) of 4-Br phenylalanine (4-BrF).

2) The peptide analog according to claim 1, wherein said peptide is further covalently linked to an acetyl group and/or an acyl group —C(O)R where R is a C₇₋₃₀ alkyl.

3) The peptide analog according to either one of claim 1 or 2, wherein said peptide covalently linked to the fluorocarbon group is no more than 13 amino acid residues.

4) The peptide analog according to any one of claims 1 to 3, wherein said fluorocarbon group linked to said peptide has the following formula (II):
CₘFₙ—C_{y}Hₓ(L) (II)
wherein m=3 to 30, n≤2m+1, y=0 to 2, x≤2y, (m+y)=3 to 30, and L, which is optional, is a linker selected from the group consisting of a PEG or a peptide having from 1 to 6 amino acids.

5) The peptide analog according to any one of claims 1 to 4, wherein said acyl group has the following formula (III):
CH₃—C_{y}Hₓ—C(O)— (III)
wherein y=7 to 30, x=2y.

6) The peptide analog according to any one of claims 1 to 5, which is selected from:
i) an apelin analog having said peptide of the formula (IV):
Xaa1-Arg-Pro-Xaa2-Leu-Xaa3-Xaa4-Xaa5-Gly-Pro-Xaa6-Pro-Xaa7 (IV)
and wherein
Xaa1 is L- or D-glutamine (QL or QD) or alanine (A);
Xaa2 is arginine (R) or lysine (K) or D-norleucine (Nle);
Xaa3 is serine (S) or alanine (A);
Xaa4 is histidine (H) or alanine (A);
Xaa5 is lysine (K) or norleucine (Nle);
Xaa6 is methionine (M), leucine (L), phenylalanine (F) or norleucine (Nle);
Xaa7 is phenylalanine (F), 4-Br phenylalanine (4-BrF), 4-(Obenzyl)phenylalanine (4-ObnF) or p-benzoyl phenylalanine (Bpa); and
ii) an apelin analog with an amino acid sequence having at least 80% identity with the sequence of (i).

7) A peptide analog according to any one of claims 1 to 5, which is selected from the group consisting of:
i) an angiotensin II analog having said peptide of the formula (V):
Xaa1-Arg-Val-Tyr-Ile-His-Pro-Xaa2 (V)
and wherein
Xaa1 is aspartic acid (D) or sarcosine;
Xaa2 is phenylalanine or OH; and
ii) an angiotensin II analog with an amino acid sequence having at least 80% identity with the sequence of (i).

8) A peptide analog according to any one of claims 1 to 5, which is selected from the group consisting of:
i) an oxytocin analog having said peptide of the formula (VI):
Cys-Tyr-Ile-Xaa1-Asp-Cys-Xaa2-Xaa3-Gly (VI)
and wherein Xaa1 is glutamine or threonine;
Xaa2 is proline or glycine;
Xaa3 is leucine, lysine or proline;
ii) an oxytocin analog with an amino acid sequence having at least 80% identity with the sequence of (i).

9) The peptide analog according to any one of claims 1 to 8, for use as a drug.

10) The peptide analog according to any one of claims 1 to 8, for use in the treatment of a GPCR-related disease selected from:
cardiovascular disease: heart failure, kidney diseases (e.g. renal failure, nephritis, etc. . . . ), hypertension, pulmonary hypertension, cirrhosis, atherosclerosis, pulmonary emphysema, pulmonary oedema, stroke, brain ischemia, myocardial impairment in sepsis;
the syndrome of inappropriate antidiuretic hormone (SIADH);
metabolic diseases: obesity, anorexia, hyperphagia, polyphagia, hypercholesterolemia, hyperglyceridemia, hyperlipemia;
various types of dementia: senile dementia, cerebrovascular dementia, dementia due to genealogical denaturation degeneratie disesases, dementia resulting from infectious diseases, deentia associated with endocrine diseases, metabolic diseases, or poisoning, dementia caused by tumors, and dementia due to traumatic diseases, depression, hyperactive child syndrome, disturbance of consciousness, anxiety disorder, schizophrenia, phobia;
pain and hyperalgesia.

11) A pharmaceutical composition comprising a peptide analog according to any one of claims 1 to 8, and one or more pharmaceutically acceptable excipient.

12) Use of a peptide analog according to anyone of claims 1 to 8 in a diagnostic method.


SEQUENCE LISTING (122 sequences)

SEQ ID NO: 1 - protein, 17 aa, Artificial Sequence
  apelin analog (formula I)
  MISC_FEATURE (3)..(3): Xaa is arginine or D-isomer arginine
  MISC_FEATURE (5)..(5): Xaa is glutamine or D-isomer glutamine
  MISC_FEATURE (9)..(9): Xaa is leucine or D-isomer leucine
  misc_feature (11)..(11): Xaa can be any naturally occurring amino acid
  MISC_FEATURE (13)..(13): Xaa is alanine or D-isomer alanine or glycine
  MISC_FEATURE (15)..(15): Xaa is methionine or norleucine
  MISC_FEATURE (17)..(17): Xaa is phenylalanine or 4-Br-phenylalanine
KFXRXRPRXSXKXPXPX

SEQ ID NO: 2 - protein, 13 aa, Artificial Sequence
  apelin analog (formula IV)
  MISC_FEATURE (1)..(1): Xaa is L- or D-glutamine or alanine
  MISC_FEATURE (4)..(4): Xaa is arginine or lysine or D-norleucine
  MISC_FEATURE (6)..(6): Xaa is serine or alanine
  MISC_FEATURE (7)..(7): Xaa is histidine or alanine
  MISC_FEATURE (8)..(8): Xaa is lysine or norleucine
  MISC_FEATURE (11)..(11): Xaa is methionine, leucine, phenylalanine or norleucine
  MISC_FEATURE (13)..(13): Xaa is phenylalanine, 4-Br-phenylalanine, 4-(Obenzyl)phenylalanine or p-benzoyl-phenylalanine
XRPXLXXXGPXPX

SEQ ID NO: 3 - protein, 8 aa, Artificial Sequence
  angiotensin II analog
  MISC_FEATURE (1)..(1): Xaa is aspartic acid or sarcosine
  MISC_FEATURE (8)..(8): Xaa is phenylalanine or OH
XRVYIHPX

SEQ ID NO: 4 - protein, 9 aa, Artificial Sequence
  oxytocin analog
  MISC_FEATURE (4)..(4): Xaa is glutamine or threonine
  MISC_FEATURE (7)..(7): Xaa is proline or glycine
  MISC_FEATURE (8)..(8): Xaa is leucine, lysine or proline
CYIXDCXXG

SEQ ID NO: 5 - protein, 15 aa, Artificial Sequence
  Balixafortide
ACSAPRYCYQKPPYH

SEQ ID NO: 6 - protein, 29 aa, Artificial Sequence
  BIS-5409 / MR-409
YADAIFTNSYRKVLGQLSARKLLQDIMSR

SEQ ID NO: 7 - protein, 20 aa, Artificial Sequence
  Bivalirudin
FPRPGGGGNGDFEEIPEEYL

SEQ ID NO: 8 - protein, 5 aa, Artificial Sequence
  BQ-123
DPVLY

SEQ ID NO: 9 - protein, 28 aa, Artificial Sequence
  Carperitide
SLRRSSCFGGRMDRIGAQSGLGCNSFRY

SEQ ID NO: 10 - protein, 18 aa, Artificial Sequence
  CEL-1000
DGQEEKAGVVSTGLIGGG

SEQ ID NO: 11 - protein, 37 aa, Artificial Sequence
  Cenderitide
GLSKGCFGLKLDRIGSMSGLGCPSLRDPRPNAPSTSA

SEQ ID NO: 12 - protein, 23 aa, Artificial Sequence
  CGEN-856S
FLGYCIYLNKRRGDPAFKRRLRD

SEQ ID NO: 13 - protein, 10 aa, Artificial Sequence
  Des-Asp Angiotensin 1
DRVYIHPFHL

SEQ ID NO: 14 - protein, 65 aa, Artificial Sequence
  Desirudin
VVYTDCTESGQNLCLCEGSNVCGQGNKCILGSDGEKNQCVTGEGTPKPQSHNDGDFEEIPEEYLQ

SEQ ID NO: 15 - protein, 4 aa, Artificial Sequence
  Elamipretide
  MISC_FEATURE (2)..(2): Xaa is 2,6-dimethyltyrosyl
RXKF

SEQ ID NO: 16 - protein, 28 aa, Artificial Sequence
  FX-06
RGHRPLDKKREEAPSLRPAPPPISGGGY

SEQ ID NO: 17 - protein, 8 aa, Artificial Sequence
  LJPC-501 (Angiotensin II)
DRVYIHPF

SEQ ID NO: 18 - protein, 37 aa, Artificial Sequence
  MP-3167 (Vastiras)
AVSNADLMDFKNLLDHLEEKMPLEDEVVPPQVLSERN

SEQ ID NO: 19 - protein, 19 aa, Artificial Sequence
  NA-1
YRKKRRQRRRKLSSIESDV

SEQ ID NO: 20 - protein, 40 aa, Artificial Sequence
  NBI-69734 (Urocortin II)
IVLSLDVPIGLLQILLEQARARAAREQATTNARILARVGH

SEQ ID NO: 21 - protein, 32 aa, Artificial Sequence
  Nesiritide
SPKMVQGSGCFGRKMDRISSSSGLGCKVLRRH

SEQ ID NO: 22 - protein, 14 aa, Artificial Sequence
  PL-3994
  MISC_FEATURE (7)..(7): Xaa is Nle
CHFAGRXDRISCYR

SEQ ID NO: 23 - protein, 44 aa, Artificial Sequence
  RGN-352 (Thymosin beta-4)
MSDKPDMAEIEKFDKSKLKKTETQEKNPLPSKETIEQEKQAGES

SEQ ID NO: 24 - protein, 17 aa, Artificial Sequence
  Solnatide
CGQRETPEGAEAKPWYC

SEQ ID NO: 25 - protein, 8 aa, Artificial Sequence
  TRV027
GRVYIHPA

SEQ ID NO: 26 - protein, 32 aa, Artificial Sequence
  Ularitide (Urodilatin)
TAPRSLRRSSCFGGRMDRIGAQSGLGCNSFRY

SEQ ID NO: 27 - protein, 17 aa, Artificial Sequence
  acALY-18
ALYDKGYTSKEQKDCVG

SEQ ID NO: 28 - protein, 12 aa, Artificial Sequence
  Cibinetide
  MISC_FEATURE (1)..(1): Xaa is 5-Oxo-
XPEQLERALDSS

SEQ ID NO: 29 - protein, 17 aa, Artificial Sequence
  COG-112
RQIKIWFQNRRMKWKKC

SEQ ID NO: 30 - protein, 39 aa, Artificial Sequence
  corticotropin
SYSMEHFRWGKPVGKKRRPVKVYPDGAEDQLAEAFPLEF

SEQ ID NO: 31 - protein, 5 aa, Artificial Sequence
  PMZ-2123
DPVLW

SEQ ID NO: 32 - protein, 40 aa, Artificial Sequence
  PT-320
SHGEGTFATSDLSKQMQQQAVRLFIQWLKDGGPSSGAPPP

SEQ ID NO: 33 - protein, 25 aa, Artificial Sequence
  Ziconotide
CKGKGAKCSRLMYDCCTGSCRSGKC

SEQ ID NO: 34 - protein, 8 aa, Artificial Sequence
  AB-103
SPMLVAYD

SEQ ID NO: 35 - protein, 30 aa, Artificial Sequence
  AG-30/5C
MLKLIFLHRLKRMRKRLKRKLRLWHRKRYK

SEQ ID NO: 36 - protein, 14 aa, Artificial Sequence
  FOL-005
VDTYDGDISWYGLR

SEQ ID NO: 37 - protein, 25 aa, Artificial Sequence
  Granexin
RQPKIWFPNRRKPWKKRPRPDDLEI

SEQ ID NO: 38 - protein, 37 aa, Artificial Sequence
  LL-37
LLGDFFRKSKEKIGKEFKRIVQRIKDFLRNLVPRTES

SEQ ID NO: 39 - protein, 12 aa, Artificial Sequence
  Omiganan
ILRWPWWPWRRK

SEQ ID NO: 40 - protein, 25 aa, Artificial Sequence
  PXL-01
EATKCFQWQRNMRKVRGPPVSCIKR

SEQ ID NO: 41 - protein, 44 aa, Artificial Sequence; identical to SEQ ID NO: 23
  RGN-137

SEQ ID NO: 42 - protein, 20 aa, Artificial Sequence
  SR-0379
MLKLIFLHRLKRMRKRLKRK

SEQ ID NO: 43 - protein, 18 aa, Artificial Sequence
  B-2088
RGRKVVRRKKRRVVKRGR

SEQ ID NO: 44 - protein, 31 aa, Artificial Sequence
  XG-102
DQSRPVQPFLNLTTPRKPRPPRRRQRRKKRG

SEQ ID NO: 45 - protein, 24 aa, Artificial Sequence
  Cosyntropin ER
SYSMEHFRWGKPVGKKRRPVKVYP

SEQ ID NO: 46 - protein, 34 aa, Artificial Sequence
  FE-203799
HGDGSFSDEMFTILDLLAARDFFINWLIQTKITD

SEQ ID NO: 47 - protein, 14 aa, Artificial Sequence
  Linaclotide
CCEYCCNPACTGCY

SEQ ID NO: 48 - protein, 8 aa, Artificial Sequence
  Octreotide
  MISC_FEATURE (6)..(6): Xaa is DL-threonine
  MISC_FEATURE (8)..(8): Xaa is DL-threonine
FCFWKXCX

SEQ ID NO: 49 - protein, 8 aa, Artificial Sequence
  Octreotide Acetate
FCFWKTCT

SEQ ID NO: 50 - protein, 16 aa, Artificial Sequence
  Plecanatide
NDECELCVNVACTGCL

SEQ ID NO: 51 - protein, 13 aa, Artificial Sequence
  Recanaclotide
  MISC_FEATURE (3)..(3): Xaa is Ser(PO3H2)
CCXLCCNPACTGC

SEQ ID NO: 52 - protein, 14 aa, Artificial Sequence
  Somatostatin
  MISC_FEATURE (10)..(10): Xaa is DL-threonine
  MISC_FEATURE (12)..(12): Xaa is DL-threonine
AGCKNFFWKXFXSC

SEQ ID NO: 53 - protein, 33 aa, Artificial Sequence
  Teduglutide
HGDGSFSDEMNTILDNLAARDFINWLIQTKITD

SEQ ID NO: 54 - protein, 33 aa, Artificial Sequence
  ZP-1848
HGEGTFSSELATILDALAARDFIAWLIATKITD

SEQ ID NO: 55 - protein, 13 aa, Artificial Sequence
  Afamelanotide
  MISC_FEATURE (4)..(4): Xaa is Nle
SYSXEHFRWGKPV

SEQ ID NO: 56 - protein, 8 aa, Artificial Sequence
  AZP-531
SPEHQRVQ

SEQ ID NO: 57 - protein, 25 aa, Artificial Sequence
  LJPC-401
DTHFPICIFCCGCCHRSKCGMCCKT

SEQ ID NO: 58 - protein, 8 aa, Artificial Sequence
  Setmelanotide
RCAHFRWC

SEQ ID NO: 59 - protein, 7 aa, Artificial Sequence
  TXA-127
DRVYIHP

SEQ ID NO: 60 - protein, 13 aa, Artificial Sequence
  AMY-101
  MISC_FEATURE (9)..(9): Xaa is Sarcosine
YICVWQDWXHRCI

SEQ ID NO: 61 - protein, 60 aa, Artificial Sequence
  Cardiotoxin
LKCNKLVPLFYKTCPAGKNLCYKMFMVSNKMVPVKRGCIDVCPKSSLLVKYVCCNTDRCN

SEQ ID NO: 62 - protein, 58 aa, Artificial Sequence
  BL-8040
RPDFCLEPPYTGPCRARIIRYFYNAKAGLCQTFVVGGCRAKRNNFKSAEDCERTCGGA

SEQ ID NO: 63 - protein, 14 aa, Artificial Sequence
  BL-8040
  MISC_FEATURE (3)..(3): Xaa is Nal
  MISC_FEATURE (6)..(6): Xaa is Cit
  MISC_FEATURE (12)..(12): Xaa is Cit
RRXCYXKKPYRXCR

SEQ ID NO: 64 - protein, 9 aa, Artificial Sequence
  CBLB-612
VQGEESNDK

SEQ ID NO: 65 - protein, 21 aa, Artificial Sequence
  EPO-018B
GGTYSCHFGALTWVCRPQRGA

SEQ ID NO: 66 - protein, 7 aa, Artificial Sequence
  Etelcalcetide Hydrochloride
CARRRAR

SEQ ID NO: 67 - protein, 301 aa, Artificial Sequence
  MOD-4023
MATGSRTSLLLAFGLLCLPWLQEGSASSSSKAPPPSLPSPSRLPGPSDTPILPQFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSSSSKAPPPSLPSPSRLPGPSDTPILPQSSSSKAPPPSLPSPSRLPGPSDTPILPQ

SEQ ID NO: 68 - protein, 16 aa, Artificial Sequence
  ATI-2341
MGYQKKLRSMTDKYRL

SEQ ID NO: 69 - protein, 19 aa, Artificial Sequence
  Edratide
GYYWSWIRQPPGKGEEWIG

SEQ ID NO: 70 - protein, 21 aa, Artificial Sequence
  Forigerimod Acetate
  MISC_FEATURE (10)..(10): Xaa is DL-threonine
RIHMVYSKRXGKPRGYAFIEY

SEQ ID NO: 71 - protein, 37 aa, Artificial Sequence
  hPTH-1-37
SVSEIQLMHNLGKHLNSMERVEWLRKKLQDVHNFVAL

SEQ ID NO: 72 - protein, 16 aa, Artificial Sequence
  ALB-408
LVRYTKKVPQVSTPTL

SEQ ID NO: 73 - protein, 36 aa, Artificial Sequence
  Enfuvirtide
YTSLIHSLIEESQNQQEKNEQELLELDKWASLWNWF

SEQ ID NO: 74 - protein, 34 aa, Artificial Sequence
  FB-006M
WEEWDREINNYTKLIHELIEESQNQQEKNEQELL

SEQ ID NO: 75 - protein, 28 aa, Artificial Sequence
  Thymalfasin
SDAAVDTSSEITTKDLKEKKEVVEEAEN

SEQ ID NO: 76 - protein, 36 aa, Artificial Sequence
  Sifuvirtide
SWETWEREIENYTRQIYRILEESQEQQDRNERDLLE

SEQ ID NO: 77 - protein, 28 aa, Artificial Sequence
  Thymalfasin
SDAAVDTSSEITTKDLKEKKEVVEEAEN

SEQ ID NO: 78 - protein, 40 aa, Artificial Sequence
  VIR-576
LEAIPCSIPPEFLFGKPFVFLEAIPCSIPPEFLFGKPFVF

SEQ ID NO: 79 - protein, 37 aa, Artificial Sequence
  AC-163794
EGTFISDYSIAMDKIHQQDFVNWLLAQKPSSGAPPPS

SEQ ID NO: 80 - protein, 32 aa, Artificial Sequence
  Calcitonin
CGNLSTCMLGTYTQDFNKFHTFPQTAIGVGAP

SEQ ID NO: 81 - protein, 7 aa, Artificial Sequence
  DD-04107
  MISC_FEATURE (1)..(1): Xaa is a palmitoyl-
XEEMQRR

SEQ ID NO: 82 - protein, 31 aa, Artificial Sequence
  Exendin-(9-39)
DLSKQMEEEAVRLFIEWLKNGGPSSGAPPPS

SEQ ID NO: 83 - protein, 14 aa, Artificial Sequence
  HIP-2B
IGLHDPTQGTEPNG

SEQ ID NO: 84 - protein, 37 aa, Artificial Sequence
  HS-20004
EGTFTSDVSSYLEEEAAKEFIAWLVRGGPSSGAPPPS

SEQ ID NO: 85 - protein, 51 aa, Artificial Sequence
  insulin aspart
  DISULFID (7)..(37)
  DISULFID (19)..(50)
  DISULFID (36)..(41)
FVNQHLCGSHLVEALYLVCGERGFFYTDKTGIVEQCCTSICSLYQLENYCN

SEQ ID NO: 86 - protein, 50 aa, Artificial Sequence
  insulin degludec
  DISULFID (7)..(36)
  DISULFID (19)..(49)
  DISULFID (35)..(40)
FVNQHLCGSHLVEALYLVCGERGFFYTPKGIVEQCCTSICSLYQLENYCN

SEQ ID NO: 87 - protein, 50 aa, Artificial Sequence; identical to SEQ ID NO: 86
  insulin detemir
  DISULFID (7)..(36)
  DISULFID (19)..(49)
  DISULFID (35)..(40)

SEQ ID NO: 88 - protein, 53 aa, Artificial Sequence
  insulin glargine
  DISULFID (7)..(39)
  DISULFID (19)..(52)
  DISULFID (38)..(43)
FVNQHLCGSHLVEALYLVCGERGFFYTPKTRRGIVEQCCTSICSLYQLENYCG

SEQ ID NO: 89 - protein, 51 aa, Artificial Sequence
  insulin glulisine
  DISULFID (7)..(37)
  DISULFID (19)..(50)
  DISULFID (36)..(41)
FVKQHLCGSHLVEALYLVCGERGFFYTPETGIVEQCCTSICSLYQLENYCN

SEQ ID NO: 90 - protein, 51 aa, Artificial Sequence
  insulin lispro
  DISULFID (7)..(37)
  DISULFID (19)..(50)
  DISULFID (36)..(41)
FVNQHLCGSHLVEALYLVCGERGFFYTKPTGIVEQCCTSICSLYQLENYCN

SEQ ID NO: 91 - protein, 51 aa, Artificial Sequence
  insulin neutral
  DISULFID (7)..(37)
  DISULFID (19)..(50)
  DISULFID (36)..(41)
FVNQHLCGSHLVEALYLVCGERGFFYTPKAGIVEQCCTSICSLYQLENYCN

SEQ ID NO: 92 - protein, 44 aa, Artificial Sequence
  Lixisenatide
HGEGTFTSDLSKQMEEEAVRLFIEWLKNGGPSSGAPPSKKKKKK

SEQ ID NO: 93 - protein, 39 aa, Artificial Sequence
  PEX-168
HGEGTFTSDLSKQMEEEAVRLFIEWLKNGGPSSGAPPPS

SEQ ID NO: 94 - protein, 22 aa, Artificial Sequence
  Pexiganan
GIGKFLKKAKKFGKAFVKLLKK

SEQ ID NO: 95 - protein, 5 aa, Artificial Sequence
  PMZ-2123
DPVLW

SEQ ID NO: 96 - protein, 34 aa, Artificial Sequence
  Teriparatide
SVSEIQLMHNLGKHLNSMERVEWLRKKLQDVHNF

SEQ ID NO: 97 - protein, 34 aa, Artificial Sequence; identical to SEQ ID NO: 96
  teriparatide acetate

SEQ ID NO: 98 - protein, 44 aa, Artificial Sequence
  tesamorelin acetate
  MISC_FEATURE (1)..(1): Xaa is N-[(3E)-1-oxo-3-hexenyl]-
XYADAIFTNSYRKLGQLSARKLLQDIMSRQQGESNQERGARARL

SEQ ID NO: 99 - protein, 5 aa, Artificial Sequence
  VTC-G15
LVKGR

SEQ ID NO: 100 - protein, 29 aa, Artificial Sequence
  ZP-4207
HSQGTFTSDYSKYLDRARADDFVAWLKST

SEQ ID NO: 101 - protein, 24 aa, Artificial Sequence
  AD-01
QIRQQPRDPPTETLELEVSPDPAS

SEQ ID NO: 102 - protein, 25 aa, Artificial Sequence
  ATSP-7041
CKGGRAKDCGGKLAKLAKKLAKLAK

SEQ ID NO: 103 - protein, 12 aa, Artificial Sequence
  BMTP-11
  MISC_FEATURE (9)..(9): Xaa is Cba
LTFEYWAQXSAA

SEQ ID NO: 104 - protein, 26 aa, Artificial Sequence
  BMTP-11
CGRRAGGSCGGDKLAKLAKKLAKLAK

SEQ ID NO: 105 - protein, 6 aa, Artificial Sequence
  Box-5
MDGCEL

SEQ ID NO: 106 - protein, 16 aa, Artificial Sequence
  Dolcanatide
NDECELCVNVACTGCL

SEQ ID NO: 107 - protein, 5 aa, Artificial Sequence
  Dusquetide
RIVPA

SEQ ID NO: 108 - protein, 6 aa, Artificial Sequence
  Foxy-5
MDGCEL

SEQ ID NO: 109 - protein, 16 aa, Artificial Sequence
  GO-2032c
RRRRRRRRRCQCRRKN

SEQ ID NO: 110 - protein, 9 aa, Artificial Sequence
  LTX-315
  MISC_FEATURE (8)..(8): Xaa id Dip
KKWWKKWXK

SEQ ID NO: 111 - protein, 8 aa, Artificial Sequence
  LY-2510924
  MISC_FEATURE (5)..(5): Xaa is Nal
FYKRXGEK

SEQ ID NO: 112 - protein, 14 aa, Artificial Sequence
  SORC-13
KIEFLHPSKVDLPR

SEQ ID NO: 113 - protein, 14 aa, Artificial Sequence
  B27-PD
ALNEDLSSWTAADT

SEQ ID NO: 114 - protein, 12 aa, Artificial Sequence
  C-16Y
DFKLFAVYIKYR

SEQ ID NO: 115 - protein, 44 aa, Artificial Sequence; identical to SEQ ID NO: 23
  RGN-259

SEQ ID NO: 116 - protein, 32 aa, Artificial Sequence
  CGEN-25009
GQKGQVGPPGAAVRRAYAAFSVGRRAYAAFSV

SEQ ID NO: 117 - protein, 21 aa, Artificial Sequence
  Lucinactant
KLLLLKLLLLKLLLLKLLLLK

SEQ ID NO: 118 - protein, 22 aa, Artificial Sequence
  MMI-0100
YARAAARQARAKALARQLGVAA

SEQ ID NO: 119 - protein, 10 aa, Artificial Sequence
  Gonadorelin
  MISC_FEATURE (1)..(1): Xaa is pyroglutamic acid
XHWSYGLRPG

SEQ ID NO: 120 - protein, 10 aa, Artificial Sequence
  Nafarelin
  MISC_FEATURE (1)..(1): Xaa is pyroglutamic acid
  MISC_FEATURE (6)..(6): Xaa is NaI
XHWSYXLRPG

SEQ ID NO: 121 - protein, 9 aa, Artificial Sequence
  Oxytocin
CYIQNCPLG

SEQ ID NO: 122 - protein, 9 aa, Artificial Sequence
  Urofollitropin
CYITNCPLG